(19) United States
(12) Patent Application Publication
(10) Pub. No.: US 2005/0209195 A1
(43) Pub. Date: Sep. 22, 2005

(54) Indolinone derivatives

(75) Inventors: Ernesto Menta, Cernusco Sul Naviglio (IT); Paolo G. Cassara, Milan (IT); Giulio Mariotti, Milan (IT); Gennaro Colella, Milan (IT); Franco Zunino, Milan (IT); Cinzia Lanzi, Milan (IT); Giuliana Cassinelli, Milan (IT); Mario Grugni, Milan (IT)

Correspondence Address: DONALD W. WYATT;CELL THERAPEUTICS, INC., 501 ELLIOTT AVENUE WEST, #400, SEATTLE, WA, 98119, US

(73) Assignee: Cell Therapeutics Europe S.r.I, Milan (IT)

(21) Appl. No.: 11/039,772

(22) Filed: Jan. 19, 2005

Related U.S. Application Data
(60) Provisional application No. 60/537,689, filed on Jan. 20, 2004.

(51) Int. Cl. A61K 31/675; A61K 31/404; C07D 43/02; C07D 209/36

(52) U.S. Cl. 514/80; 514/414; 514/418; 548/465; 548/484

(57) ABSTRACT

Novel derivatives of compound (E)-1,3-dihydro-5,6-dimethoxy-3-[(4-hydroxyphenyl)methylene]-2H-indol-2-one and the use thereof for the preparation of medicaments for the treatment of tumors in which the tyrosine kinase activity proteins Met, PDGF-R, FGF-R1, FGF-R3, Kit and the oncoproteins of the Ret family are involved.

The present invention relates to novel (E)-1,3-dihydro-5,6-dimethoxy-3-[(4-hydroxyphenyl)methylene]-2H-indol-2-one derivatives useful for the preparation of medicaments for the treatment of tumors in which the proteins with tyrosine kinase activity Met, PDGF-R, FGF-R1, FGF-R3, Kit and oncoproteins of the Ret family (receptors constitutively active following mutation) are involved.

### TECHNOLOGICAL BACKGROUND

A variety of compounds with 2-indolinone structure have numerous pharmacological activities. In particular, in view of the tyrosine kinase activity modulation properties exhibited by 2-indolinone derivatives, the use thereof for the treatment of different pathologies such as cancer, mastocytosis, diabetes, allergic chronic rhinitis, autoimmune diseases, restenosis, fibrosis, psoriasis, von Hippel-Lindau syndrome, osteoarthritis, rheumatoid arthritis, angiogenesis, inflammatory, immunological and cardiovascular disorders, has been suggested (WO 01/45689, WO 01/60814, WO 99/48868, U.S. Pat. Nos. 6,316,429, 6,316,635, 6,133,305 and 6,248,771). 2-Indolinone derivatives substituted at the 3-position with an arylidene residue are disclosed in the following U.S. Pat. Nos. 6,531,502, 6,429,389, 6,469,032, 6,268,391, 6,248,771, 6,225,335, 5,886,020, 5,883,116, 5,834,504, 5,502,072, 5,883,113, 6,147,106, 5,382,593, 5,124,347, U.S. patent application Ser. Nos. 2002/0102608, 2002/0015938, 2001/0027207, 2002/0187978. 2-Indolinone derivatives inhibiting protein kinases of the c-kit family are disclosed in U.S. patent application Ser. No. 2004/0002534.

Oncogenes RET/PTC are mainly expressed in thyroid papillary tumors and derive from somatic chromosomal rearrangements in which the proto-RET gene is involved. Protein Ret/ptc1 is a fusion protein produced by the oncogene RET/PTC1, deriving from the rearrangement of the tyrosine kinase of the proto-RET gene (normal) with H4/D10S170 gene. The products of said rearranged genes are characterized by tyrosine kinase activity independent of the ligand and cytoplasmic localization.

Int. J. Cancer 85, 384-390 (2000) discloses the inhibiting effect on oncoprotein Ret/ptc1 tyrosine kinase activity by arylidene-2-indolinone derivatives. 1,3-Dihydro-5,6-dimethoxy-3-[(4-hydroxyphenyl)methylene]-2H-indol-2-one, which is capable of restoring the normal phenotype in NIH3T3 cells transformed by oncoprotein Ret/ptc1, is mentioned among the mainly active compounds. The same paper suggests the use of arylidene-2-indolinone tyrosine kinase inhibitors in the study of Ret signalling and in the control of cell proliferation in pathologies related to Ret and Ret/ptcs.

PCT/EP03/07963, filed on Jul. 22, 2003, discloses that (E)-1,3-dihydro-5,6-dimethoxy-3-[(4-hydroxyphenyl)methylene]-2H-indol-2-one effectively inhibits proteins endowed with tyrosine kinase activity, different from Ret/ptc1, which play an important role in the onset, progression and diffusion of tumors. More particularly, (E)-1,3-dihydro-5,6-dimethoxy-3-[(4-hydroxyphenyl)methylene]-2H-indol-2-one proved capable of fully inhibiting self-phosphorylation of tyrosine kinases Ret/MEN2A (mutC634R and mutC634W), Ret/MEN2B (mutM918T), Met, PDGF-R, FGF-R1, FGF-R3 and Kit (c-Kit and mutΔ559), of down regulating their expression and of restoring the phenotype of cells transformed by them.

### DISCLOSURE OF THE INVENTION

It has now been found that novel 1,3-dihydro-5,6-dimethoxy-3-[(4-hydroxyphenyl)methylene]-2H-indol-2-one derivatives exert inhibiting activity towards the proteins with tyrosine kinase activity Met, PDGF-R, FGF-R, Kit and oncoproteins of the Ret family. These proteins play an important role in tumor genesis, progression and metastatization. The compounds of the invention have the following general formula (I):

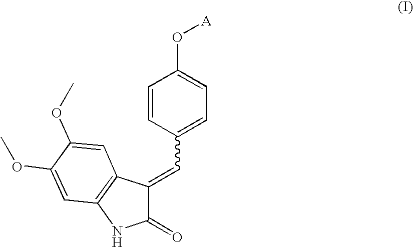

or pharmaceutically acceptable thereof, stereomeric or tautomeric forms, in which:

A is selected from the group consisting of: —R, —C(═O)—R1, —C(═O)—OR2, —C(═X)—NR3R4, —P(═OR)(OR5)(OR6), —(O═S═O)R7; or A is an optionally protected aminoacyl residue;

R is optionally substituted alkyl, optionally substituted alkenyl, optionally substituted alkynyl;

R1 is H, optionally substituted alkyl, optionally substituted alkenyl, optionally substituted alkynyl, optionally substituted carbocyclyl, optionally substituted heterocarbocyclyl;

R2 is optionally substituted alkyl, optionally substituted alkenyl, optionally substituted alkynyl, optionally substituted carbocyclyl, optionally substituted heterocarbocyclyl;

R3 and R4 are independently H, optionally substituted alkyl, optionally substituted alkenyl, optionally substituted alkynyl, optionally substituted carbocyclyl, optionally substituted heterocarbocyclyl, or R3 and R4, together with the nitrogen atom they are linked to, form a heterocyclic ring which can be optionally substituted;

R5 and R6 are independently H, C1-C6-alkyl, C7-C10-aralkyl;

R7 is C1-C6-alkyl, C7-C10-aralkyl;

X is O, S;

and the pharmaceutically acceptable salts thereof, stereomeric or tautomeric forms thereof.

In the formula (I), the line [symbol] means that the compounds of the invention can exist as geometric isomers around the double bond present at the 3-position of the indolinone ring. The present invention also relates to the single stereoisomers of the compounds of formula (I), represented by Formula I-(E) and Formula I-(Z):

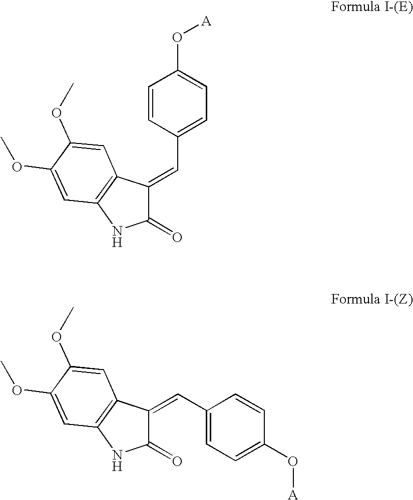

wherein A is as defined in formula (I),

and the mixtures thereof.

The expression “optionally substituted alkyl” preferably means an alkyl group C1-C10 optionally substituted with hydroxy, alkoxy, amino, mono-alkylamino, di-alkylamino, carboxy, alkyloxycarbonylamino groups or interrupted by one to three oxygen or nitrogen atoms. Examples of said groups comprise methyl, ethyl, n-propyl, isopropyl, tert-butyl, n-butyl, n-pentyl, n-hexyl, 2-hydroxyethyl, 2-aminoethyl, 2-dimethylaminoethyl, carboxymethyl, 2-hydroxyethoxy, 4-tert-butoxycarbonylamino-butyl, 2-(2-methoxy-ethoxy)ethoxy methyl.

The expression “optionally substituted alkenyl” preferably means a C2-C6 alkenyl group optionally substituted with hydroxy, amino, mono-alkylamino, di-alkylamino groups. Examples of said groups comprise ethenyl, allyl.

The expression “optionally substituted alkynyl” preferably means a C2-C6 alkynyl group optionally substituted with hydroxy, amino, mono-alkylamino, di-alkylamino groups. Example of said groups comprise ethynyl, 2-propynyl.

The expression “optionally substituted carbocyclyl” preferably means a C3-C7-cycloalkyl group optionally substituted with one or more hydroxy, amino, mono-alkylamino, di-alkylamino, C1-C3-alkoxy groups or an aryl group, in particular phenyl, optionally substituted with one or more hydroxy, amino, C1-C3-alkoxy, nitro, halogen, C1-C3-alkyl, haloalkyl C1-C3, C1-C3 haloalkoxy, cyano groups, optionally substituted heterocarbocyclyl, preferably, methoxy, nitro. Examples of said groups comprise cyclopentyl, cyclohexyl, cyclopropyl, phenyl, 4-methoxy-phenyl, 4-nitrophenyl, 4-cyanophenyl, 4-(1-tetrazolyl)phenyl.

The expression “optionally substituted heterocarbocyclyl” preferably means a 5-7 membered saturated or unsaturated heterocyclic group containing one to four heteroatoms selected from nitrogen, oxygen or sulfur, optionally substituted at the nitrogen or at the carbon ring atoms by C1-C6 alkyl, phenyl or heterocyclic groups as defined above. Examples of said groups comprise N-methyl-2-pyrrolyl, 2-, 3- or 4-pyridyl, N-morpholinyl, N-piperazinyl, 5-oxazolyl, 4-methyl-piperazinyl, 4-phenyl-piperazinyl, 4-(2-hydroxyethyl)-piperazinyl, 4-(2-hydroxyethoxy-ethyl)-piperazinyl, N-pyrrolidinyl, 4-(1′-piperidino)-1-piperidinyl.

C7-C10-aralkyl is preferably benzyl.

When R3 and R4 taken together form an optionally substituted heterocyclic ring, this is preferably a piperazinyl, morpholinyl, piperidinyl, N-methyl-piperazinyl, N-phenyl-piperazinyl, 4-piperidino-piperidinyl, N-(2-hydroxyethyl) piperazinyl, N-(2-hydroxyetoxyethyl) piperazinyl ring.

R is preferably 2-hydroxyethyl, 2-aminoethyl, tert-butoxycarbonylmethyl or carboxymethyl.

R1 is preferably methyl, tert-butyl, n-nonyl, adamantyl, 2-(2-methoxy-ethoxy)ethoxymethyl, phenyl, 4-cyano-phenyl, 4-(1-tetrazolyl)-phenyl, 3-pyridyl, 5-oxazolyl, 2-pyrazinyl, 1-methyl-1H-2-pyrrolyl, 4-tert-butoxycarbonylaminobutyl, 2(S)-tert-butoxycarbonylaminoethyl.

R2 is preferably 4-metoxyphenyl, n-pentyl, 4-nitrophenyl.

When A is a CONR3R4 group, one of R3 and R4 is preferably hydrogen and the other is tert-butyl, n-pentyl, n-hexyl, phenyl, 4-methoxy-phenyl, or R3 and R4 are both 2-hydroxyethyl or taken together form a 4-methyl-piperazinyl, morpholyl, 4-(2-hydroxyethyl)-piperazinyl, 4-(2-(hydroxyetoxyethyl)piperazinyl, 4-(1′-piperidinyl)-piperidinyl ring.

R5 and R6 are preferably both hydrogen, benzyl or ethyl.

R7 is preferably benzyl.

The compounds of formula (I) in which A is a group of formula —C(═X)—NR3R4 being X, R3 and R4 as defined in formula (I), with the exception that R3 and R4 are different from H; can be prepared by reaction of the compound (E)-1,3-dihydro-5,6-dimethoxy-3-[(4-hydroxyphenyl)-methylene]-2H-indol-2-one with a isocyanate or a isothiocyanate of formula (II)
X═C═N—R′4 (II)

in which X is O, S and R′4 has the same meanings as R4 in formula (I), except for the meaning of H.

The compounds of formula (II) are known compounds or can be prepared with known methods (Tetrahedron Lett., 40, 1999, 2895-2898; J. Org. Chem., 61, 1996, 3929-3934; J. Med. Chem., 42, 1999, 593-600; Chem. Pharm. Bull.; 44; 11; 1996; 2042-2050; Synthesis; 11; 1982; 969-970; J. Org. Chem.; 64; 19; 1999; 6984-6988; Tetrahedron Lett.; 38; 50; 1997; 8743-8744; J. Med. Chem.; 28; 12; 1985; 1925-1933; Angew. Chem.; 34; 1921; 231.). Many of them are commercially available.

Alternatively, the compounds of formula (I) in which A is a group of formula —C(═X)—NR3R4, in which, X═O can be prepared by reacting compound (E)-1,3-dihydro-5,6-dimethoxy-3-[(4-hydroxyphenyl)methylene]-2H-indol-2-one with a 4-nitrophenyl haloformate of formula (III)

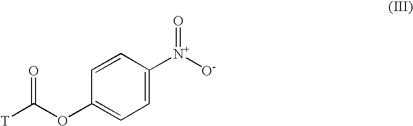

in which T is a halogen, preferably chlorine, to give the compound of formula (IV)

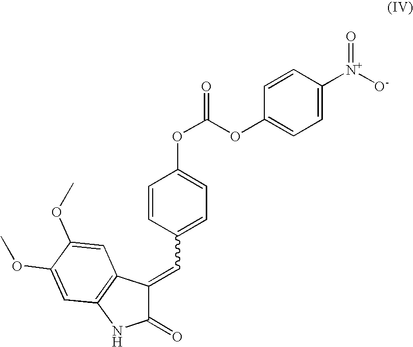

which is subsequently reacted with an amine of formula (V)
R3R4—NH₂ (V)

to give a compound of formula (I) in which R1 is a group of formula —C(═X)—NR3R4, in which, X═O

4-Nitrophenyl haloformates of formula (III) are known compounds. In particular, 4-nitrophenyl chloroformate is a commercially available compound.

The compounds of formula (I) in which A=—C(═O)—OR2, being R2 as defined in formula (I), can be prepared by reacting compound (E)-1,3-dihydro-5,6-dimethoxy-3-[(4-hydroxyphenyl)methylene]-2H-indol-2-one with haloformate of formula (VI)
T-C(═O)—OR2 (VI)

wherein R2 is as defined in formula (I).

The haloformates of formula (VI) are known compounds or can be prepared with known methods (J. Med. Chem.; 13; 1970; 1176-1179; J. Org. Chem.; 43; 1978; 2410-2414; J. Org. Chem.; 25; 1960; 1118-1123; Bioorg. Med. Chem. Lett.; 7; 8; 1997; 1071-1076; J. Med. Chem.; 43; 3; 2000; 475-487). Many of them are commercially available.

The compounds of formula (I) in which A is a —C(═O)—R1 group, being R1 as defined in formula (I), can be prepared by reacting compound (E)-1,3-dihydro-5,6-dimethoxy-3-[(4-hydroxyphenyl)methylene]-2H-indol-2-one with an acyl halide of formula (VII)
T-C(═O)—R1 (VII)

wherein T is a halogen and R1 as defined in formula (I).

Alternatively, the compounds of formula (I) in which A is a group —C(═O)—R1, in which R1 is as defined in formula (I), can be prepared by reacting compound (E)-1,3-dihydro-5,6-dimethoxy-3-[(4-hydroxyphenyl)methylene]-2H-indol-2-one with a carboxylic acid of formula (VIII)
R1—COOH (VIII)
in the presence of dehydrating agents such as carbodiimides, for example dicyclohexylcarbodiimide or 1-(3-dimethylpropyl)-3-ethylcarbodiimide, and of organic bases such as 4-dimethylaminopyridine.

The compounds of formula (I) in which A is a group of formula R, in which R is as defined in formula (I), can be prepared by reaction of a compound of formula (IX)

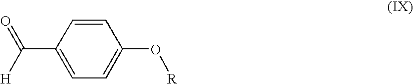

with the compound 5,6-dimethoxy-1,3-dihydro-indol-2-one of formula (X)

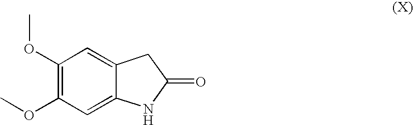

according to the conventional conditions of Knoevenagel condensation.

The reaction is generally carried out in an alcoholic solvent, for example ethanol, in the presence of traces of organic bases, for example piperidine.

The compounds of formula (IX) can be obtained by reacting 4-hydroxybenzaldehyde of formula (XI)

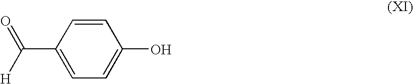

with a compound of formula R-T, in which R is as defined in formula (I) and T is a halogen, according to conventional alkylation conditions of a phenol group. The reaction is generally carried out in a solvent such as dimethylformamide, dimethylacetamide, in the presence of inorganic bases such as alkali or alkaline-earth metal hydrides or carbonates.

The synthesis of 5,6-dimethoxy-1,3-dihydro-indol-2-one of formula (X) is disclosed in PCT/EP03/07963.

The compounds of formula (I) in which A is a group of formula —P(═O)(OR5)(OR6) and R5 and R6 are C1-C4 alkyl can be prepared by reacting compound (E)-1,3-dihydro-5,6-dimethoxy-3-[(4-hydroxyphenyl)-methylene]-2H-indol-2-one with a compound of formula (XII)
T-P(═O)(OR′5)(OR′6) (XII)

in which T is a halogen and R′5 and R′6 have the same meanings as R5 and R6 in formula (I) except for the meaning of hydrogen, to give a compound of formula (I′)

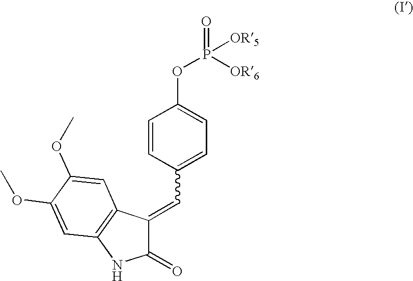

in which R′5 and R′6 are as defined in formula (XII).

The compounds of formula (I) in which A is a group of formula

—P(═O)(OR)(OR) and R5 and R6 are H can be prepared by reaction of the compound of formula (I″)

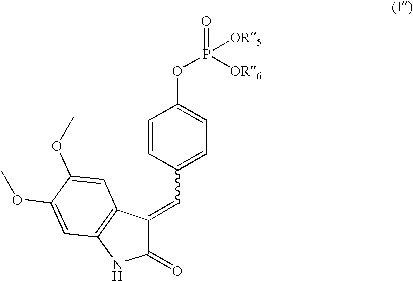

in which R″5 and R″6 are un C7-arylalkyl with a trialkylsilyl chloride of formula (XIII)
(alkyl)₃SiCl (XIII)

in the presence of an alkali metal iodide, followed by acidification with a mineral acid. The reaction is preferably carried out in a halogenated solvent, for example dichloromethane, using trimethylsilyl chloride in the presence of sodium iodide, followed by acidification with concentrated hydrochloric acid to give (E)-1,3-dihydro-5,6-dimethoxy-3-[(4-hydroxyphenyl)methylene]-2H-indol-2-one phosphoric monoester.

Other methods useful for the preparation of the compounds of formula (I) in which R1 is a group of formula —P(═O)(OR5)(OR6) are described in Journal of Medicinal Chemistry, 2000, 43, 2731-2737 or in Journal of American Chemical Society 1938, 60, 750-751, which disclose, respectively, the reaction of a phenol hydroxyl with dibenzyl phosphite followed by removal of the benzyl groups with trimethylsilyl iodide and neutralization of the resulting phosphoric acid with sodium methoxide in methanol, or by the reaction of a phenol with POCl₃ followed by hydrolysis of the resulting monophenylphosphoryl chloride to give a mono-phenylphosphoric acid and final neutralization to give the disodium mono-phenylphosphate.

The compounds of formula (I) in which A is a group of formula —(O═S═O)—R7, being R7 as defined in formula (I), can be prepared by reaction of a compound of formula (XIV)

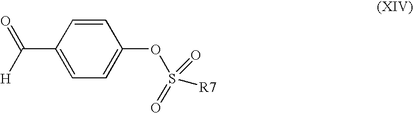

with the compound 5,6-dimethoxy-1,3-dihydro-indol-2-one of formula (X)

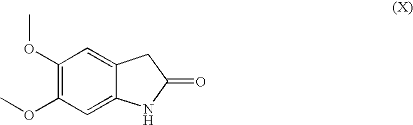

according to the conventional conditions for the Knoevenagel condensation.

The compounds of formula (XIV) can be obtained by reacting 4-hydroxybenzaldehyde of formula (XI)

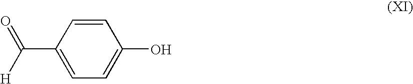

with a compound of formula R7—SO2—T in which R7 is as defined in the formula (I) and T is a halogen, according to the conventional conditions for the sulfonylation of a phenol group, well known to those skilled in the art. The reaction is usually carried out in a solvent such as dimethylformamide, dimethylacetamide, methylene chloride in the presence of inorganic bases, such as alkali or alkaline-earth metals hydrides or carbonates, or in the presence of tertiary organic bases, such as triethylamine.

The compounds of the invention exert inhibiting activity towards proteins with tyrosine kinase activity, in particular towards proteins Met, PDGF-R, FGF-R1, FGF-R3, Kit and the oncoproteins of the Ret family.

Ret oncoproteins whose receptor function is constitutively activated due to amino acid replacement are found in sporadic medullary thyroid carcinoma (MTC) and in hereditary multiple endocrine neoplasia syndrome of type 2 (MEN2A, MEN2B and MTC of family type), all characterized by the onset of MTC (Jhiang S. M. et al., Oncogene 19, 5590, 2000). While in thyroid medullar carcinoma of sporadic type RET mutations are somatic, in MEN2 patients RET mutations are present at the germinal level. These mutations induce constitutive activation of the receptor without modifying its localization in the cell membrane.

The inhibiting activity of the compounds of the invention on Ret proteins can be determined using Ret oncoproteins with Cys634 (referred to as Ret/MEN2A^{C634R} and Ret/MEN2A^{C634W}) or Met918 (referred to as Ret/MEN2B^{M918T}) mutations, which represent the RET oncoproteins more frequently expressed in MEN2A and MEN2B, respectively. The inhibiting activity of the compounds of the invention can be shown in murine cells transfected with the RET/MEN2A(C634R) gene (NIH3T3^{MEN2A(C634R)} cells) and in thyroid medullar carcinoma TT and MZ-CRC-1 cell lines, the latter characterized by the expression of Ret/MEN2A(C634W) and Ret/MEN2B(M918T), respectively. The compounds of the invention are capable of inducing in these cell lines reduction of the oncoprotein phosphorylation and expression. Furthermore, the compounds of the invention induce an antiproliferative effect due to inhibition of self-phosphorylation of the Ret/MEN2A and RET/MEN2B receptors. Evaluation of the antiproliferative effect can be carried out on NIH3T3^{MEN2A(C634R)} cells. After treatment with the compounds of the invention, these cells show regression of the transformed phenotype.

Therefore the inhibiting activity of the compounds of the invention on Ret oncoproteins suggests that they can be useful in the treatment of thyroid medullar tumours, pheochromocytoma and parathyroid hyperplasia associated with MEN2 syndrome.

Met hepatocytes growth factor receptor, is a tyrosine kinase involved in the invasive process characteristic of tumor progression and metastatic growth (Maulik G. et al., Cytok. Growth factor Rev. 13, 41, 2000). Alterations such as mutations, over-expression and involvement in autocrine loops are the cause of kinase constitutive activation. Dysregulation of Met kinase activity can be observed in a number of epithelial tumors and may be the basis of the metastatic behaviour. Met is frequently over-expressed in thyroid papillary tumors. The compounds of the invention are capable of inhibiting Met self-phosphorylation in thyroid papillary carcinoma cell lines, for example in TPC-1 cells. Inhibition of Met activity is useful in adjuvant therapy in order to reduce epithelial tumors invasivity. In connection with Met activating mutations, the compounds of the invention can have specific indications in the therapy of renal tumors.

Other tyrosine kinases, such as PDGF-R (Rosenkranz S. and Kazlauskas A., Growth factors 16, 201, 1999) and FGF-R1 (Powers C. J. Et al., Endocr. Rel. Cancer 7, 165, 2000) which are involved in autocrine loops or in neoangiogenetic processes, play an important role in tumor growth. Dysregulated activation of these receptors is observed in tumors which are unresponsive to conventional therapies, such as gliomas and melanomas. The compounds of the invention inhibit phosphorylation of the receptor of these kinase induced by autocrine stimulation or by an exogenous ligand. Therefore, the therapeutical use of the compounds of the invention in some tumours unresponsive to conventional therapies, such as gliomas and dermatofibrosarcoma protuberans, as well as the use thereof for the control of solid tumors neoangiogenesis, can be envisaged.

Activating mutations of tyrosine kinase FGF-R3 receptor such as chromosomal translocation or point mutations produce constitutively active FGF-R3 receptors which are involved in multiple myeloma and bladder and cervix carcinoma (Powers C. J. et al. Endocr. Rel. Cancer, 7, 165, 2000). The compounds of the invention inhibit tyrosine self phosphorylation and expression of FGF-R3 receptor exogenously expressed in cell systems such as NIH3T3 fibroblasts. The compounds of the invention can therefore be used in the treatment of multiple myeloma and bladder and cervix carcinomas.

Kit tyrosine kinase is constitutively activated following mutations or involvement in autocrine loops. Kit inhibition can be exploited for the treatment of gastroenteral tract stromal tumors (GIST), in small cells lung tumors, in seminomas and in some leukemias such as mastocytosis and acute myeloid leukemia (Heinrich M. C. et al., J. Clin. Oncol., 20, 1692, 2002). The compounds of the invention inhibit phosphorylation of the c-Kit human receptor in an in vitro assay such as DELFIA™ (Dissociation Enhanced Time-Resolved Fluorometric Assay) Tyrosine Kinase Assay™. Moreover, the compounds of the invention inhibit phosphorylation and expression of mutant Kit exogenously expressed in cell systems such as NIH3T3 fibroblasts. Furthermore, the compounds of the invention inhibit c-Kit activated through autocrine loops in cell systems.

Moreover, the compounds of the invention are useful in the treatment of melanomas and gliomas in which high expression of FGF-R and of the respective ligand bFGF (even when they are involved in autocrine loops) is observed. As said receptor plays a key role in neoangiogenesis, their inhibition suggests possible therapeutic intervention through inhibition of tumor vascularization.

The word “tumor” herein includes, without limitations, abnormal cell proliferation of malignant or non-malignant cells of various tissues and/or organs such as muscle, bony or connective tissue, skin, brain, lungs, genital organs, lymphatic and renal system, mammary or hematic cells, liver, digestive system, pancreas, thyroid and adrenergic glands. Abnormal cell proliferation may include, without limitations, ovary, breast, brain, prostate, colon, liver, lung, uterus, cervix, pancreas, gastrointestinal tract, head, neck, rhinopharynx, skin, bladder, stomach, kidney, or testes tumours, Kaposi sarcoma, cholangiocarcinoma, choriocarcinoma, neuroblastoma, Wilms' tumor, Hodgkin's lymphoma, melanoma, multiple myeloma, chronic lymphocytic leukemia, and acute or chronic granulocytic lymphoma.

For the envisaged therapeutic uses, the compounds of the invention and the pharmaceutically acceptable acid salts thereof will be formulated with pharmaceutically acceptable carriers and excipients. The pharmaceutical compositions will be in form suited to the oral, parenteral, sublingual or transdermal administration, preferably in the form of tablets, capsules, granules, powders, syrups, solutions, suspensions, suppositories, controlled-release forms.

These pharmaceutical preparations can be prepared with conventional procedures using ingredients known in the pharmaceutical technique. Although dosage can range depending on the severity of the disease, age of the patient, type and route of administration, the amount will usually range from 0.1 to 1000 mg/kg, preferably from 5 to 300 mg/kg, most preferably from 20 to 200 mg/kg, in a single or multiple dose, for one or repeated daily administrations.

The compounds of the invention can be administered alone or in combination with other anti-tumoral or anti-cancer agents, such as adriamycin, daunomycin, methotrexate, vincristine, 6-mercaptopurine, cytosine arabinoside, cyclophosphamide 5-FU, hexamethylmelamine, carboplatin, cisplatin, idarubicin, paclitaxel, docetaxel, topotecan, irinotecan, gemcitabine, L_PAM, BCNU, VP-16. The compounds of the invention can also be included in a kit for the treatment of tumors. The kit can include other anti-tumoral or anti-cancer agents.

The present invention will be further described in the following examples.

### EXPERIMENTAL SECTION

¹NMR Spectra were obtained with a Brucker AV spectrometer operating at 400-MHz and using deuterated DMSO as the solvent, unless otherwise specified. The purity of the compounds was evaluated by HPLC using the following operative conditions:

HPLC Operative Conditions.

| | |
|---|---|
| Column: | ZORBAX XDB C8(2) 150 × 4.6 5 μm |
| Flow: | approx. 1 ml/min. Volume injection: 5-20 μl. |
| | Detection: UV a 275 nm |
| Mobile phase: | PHASE A: KH₂PO₄ 0.02 M at pH = 2.5 with |
| | conc. Phosphoric acid |
| | PHASE B: CH₃CN(H₂O (9 ÷ 1) |
| | Gradient profile |

| Time (min) | % A |
|---|---|
| 0 | 90 |
| 15 | 60 |
| 20 | 35 |
| 35 | 35 |

Preparation of (E)-1,3-dihydro-4-hydroxybenzylidene-5,6-dimethoxy-(1H)-indol-2-one

1) Synthesis of 2-nitro-4,5-dimetoxyphenylacetic Acid

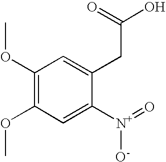

C₁₀H₁₁NO₆, M.W. 241,20

3,4-Dimetoxyphenylacetic acid (45 g, 0.23 moles, 1 eq.) was dissolved in glacial acetic acid at 28° C.-35° C., (100 mL, 2.2 volumes) under N₂ atmosphere and with mechanical stirring. The solution was cooled to 15-20° C. and a mixture of fuming nitric acid (98%, 33 mL) in glacial acetic acid (25 mL) was added in 45′. After completion of the addition, a red solid precipitated. The suspension was poured into ice-H₂O (600 mL) and kept under stirring for 2 h. The solid was filtered, washed with H₂O and dried at 60° C. for 8 h. to obtain 44 g of the desired product.

Yield 79.3% (mmoles/mmoles)

TLC (SiO₂; ethyl acetate 10/AcOH 0.5) Rf_{acid}=0.6; Rf_{product}=0.5

M.p.: 199-202° C.

¹H-NMR, (DMSO): 3.9 ppm (s, 6H); 4.0 ppm (s., 2H); 7.12 ppm (s., 1H); 7.7 ppm (s., 1H).

2) Synthesis of 1,3-dihydro-5,6-dimethoxy-(1H)-indol-2-one

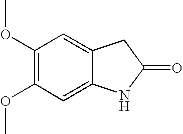

C₁₀H₁₁NO₃, M.W. 193,11

3,4-Dimethoxy-2-nitro-phenylacetic acid (9.2 g, 38.14 mmoles, 1 eq.) was suspended in glacial acetic acid (92 mL, 10 volumes), at 25° C., under N₂ atmosphere and with mechanical stirring. The resulting suspension was added with powder Fe°, 325 mesh, 97%, (12 g, 214.86 mmoles, 5.6 eq.) in two equal portions, the first portion at r.t. The mixture was refluxed and after 30′ the Fe° second portion was added. After 30′ the reaction was complete, TLC (SiO₂; CHCl₃ 9/MeOH 1), Rfₙᵢₜᵣₒ=0.65, Rf_{product}=0.71.

The gray suspension was cooled to room temperature, and acetic acid was evaporated off under reduced pressure to obtain a crude solid, which was suspended in chloroform (200 mL). The salts were filtered off and the organic phase was washed with a NaCl saturated solution (100 mL), dried over Na₂SO₄ and evaporated to dryness. The solid was suspended in ethyl ether (35 mL) for 30′, filtered and dried in a static dryer at 50° C. for 2 h., thereby obtaining 6.7 g of a beige solid.

Yield 90.9% (mmoles/mmoles)

M.p.: 199-201° C.

¹H-NMR, (DMSO): 3.4 ppm (s., 2H); 3.69 ppm (s., 3H); 3.72 ppm (s., 3H); 6.49 ppm (s., 1H); 6.92 ppm (s., 1H); 10.15 ppm (s., 1H).

3) Synthesis of (E)-1,3-dihydro-4-hydroxybenzylidene-5,6-dimethoxy-1H-indol-2-one

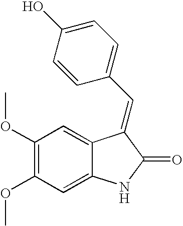

C₁₇H₁₅NO₄, M.W. 297,31

1,3-Dihydro-5,6-dimethoxy-(1H)-indol-2-one, (6.7 g, 36.9 mmoles, 1 eq.) was dissolved in dry DMSO (50 mL), at room temperature. The solution was added with 4-hydroxybenzaldehyde (5.41 g, 44.3 mmoles, 1.2 eq.) and piperidine (4.38 g, 44.3 mmoles, 1.2 eq.). The mixture was kept under stirring for 16h. The mixture was poured into H2O (250 mL) and HCl 0.5N (150 mL). The precipitated solid was cooled to 5-10° C. for 1 h, filtered and dried under vacuum at 80° C. for 2 h. 13 g of a wet solid were obtained, which were crystallized from absolute ethanol, to obtain 6.77 g of product.

Yield 61.6% (mmoles/mmoles)

M.p.: 238-240° C.

Rf (silica; ethyl acetate 100%)=0.68

¹H-NMR, (DMSO): 3.6 ppm (s., 3H); 3.8 ppm (s., 3H); 6.5 ppm (s., 1H); 6.9 ppm (d., 2H, J=8.6 Hz); 7.25 ppm (s., 1H); 7.38 ppm (s., 1H); 7.6 ppm (d., 2H, J=8.6 Hz); 10 ppm (broad s.); 12.8 ppm (s.).

The E stereochemistry of the exocyclic double bond at the 2-position was ascribed on the basis of 1D NOE NMR experiments.

### EXAMPLES A-B

### Synthesis of the Compounds of the Invention in which A is a Group of Formula —C(═X)—NR3R4

Scheme 1 summarizes the two general methods (Method I and Method II) which can be used for the preparation of these products. The compounds prepared with said methods are reported in Table 1.

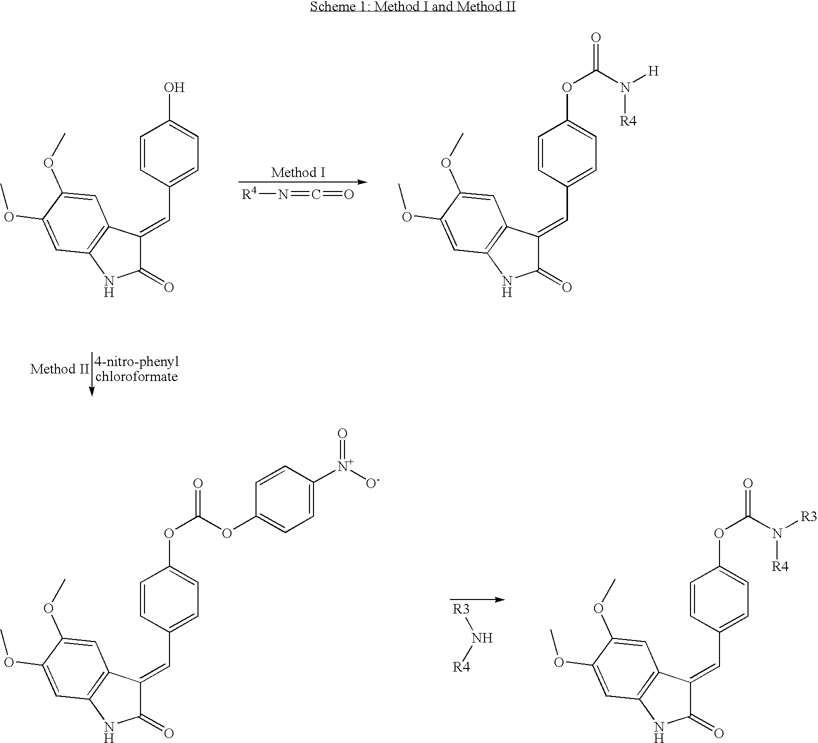

TABLE 1

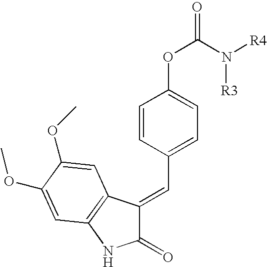

| Compound | NR3R4 | Method |
|---|---|---|
| A.1 | 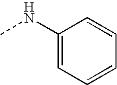 | I |
| A.2 | 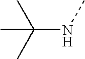 | I |
| A.3 | 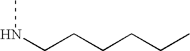 | I |
| A.4 | 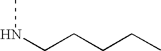 | I |
| A.5 | 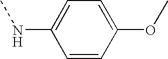 | I |
| B.1 | 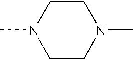 | II |
| B.2 | 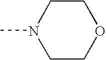 | II |
| B.3 | 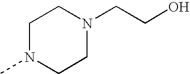 | II |
| B.4 | 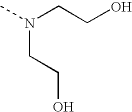 | II |
| B.5 | 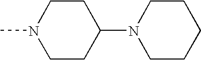 | II |
| B.6 | 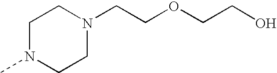 | II |
| B.7 | 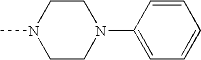 | II |

Method I

A.1 Synthesis of Phenylcarbamic Acid, (E)-4-(5,6-dimethoxy-2-oxo-1,2-dihydro-indol-3-ylidenemethyl)phenyl Ester.

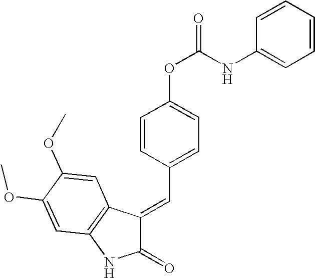

A suspension of (E)-1,3-dihydro-4-hydroxybenzylidene-5,6-dimethoxy-(1H)-indol-2-one. (100 mg, 0.33 mmoles) and TEA (0.06 ml, 0.4 mmoles) in 10 ml of dichloromethane (DCM), under nitrogen stream, was dropwise added with phenyl isocyanate (0.03 ml, 0.4 mmoles), stirring at room temperature. After 1 h stirring was interrupted and a TLC control (eluent AcOEt÷n-hexane 8÷2) was performed. The formed precipitate was filtered and washed with 2 ml of DCM. Recrystallization with 50 ml of isopropanol afforded 80 mg of product (0.192 mmoles, yield 58.2%).

M.p.: 201-203° C.; 1H NMR (DMSO-d6+TFA) δH: 3.61 (3H, s), 3.82 (3H, s), 6.51 (1H, s), 7.09 (1H, t), 7.11 (1H, s), 7.35 (2H, t), 7.41 (2H, d, J=9 Hz), 7.49 (1H, s), 7.54 (2H, d, J=9 Hz), 7.81 (2H, d).

HPLC purity: 93.8%. Elemental analysis (C24H20N2O5): found C (68.38%), H, (4.94%), N, (6.60%)—calculated C (69.22%), H, (4.84%), N, (6.73%).

According to the procedure of Example A.1 (Method I), the following compounds were prepared (Table 2):

TABLE 2

| | | |
|---|---|---|
| A.2 | tert-Butyl carbamic acid, (E) 4-(5,6-di- methoxy-2-oxo-1,2-dihydro-indol-3-yl- idenemethyl)phenyl ester | 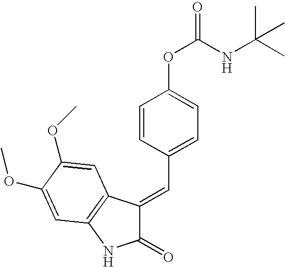 |
| A.3 | n-Pentyl carbamic acid, (E)-4-(5,6-di- methoxy-2-oxo-1,2-dihydro-indol-3-yl- idenemethyl)phenyl ester | 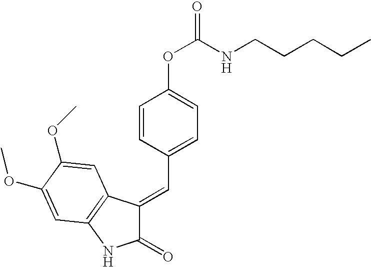 |
| A.4 | n-Hexyl carbamic acid, (E)-4-(5,6-di- methoxy-2-oxo-1,2-dihydro-indol-3-yl- idenemethyl)phenyl ester | 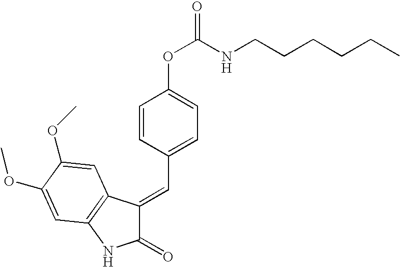 |
| A.5 | 4-Methoxyphenyl-carbamic acid, (E)-4-(5,6-di- methoxy-2-oxo-1,2-dihydro-indol-3-yl- idenemethyl)phenyl ester | 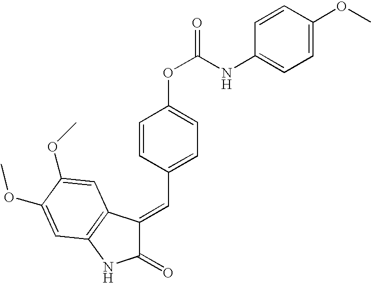 |

A.2 tert-Butyl Carbamic Acid, (E)-4-(5,6-dimethoxy-2-oxo-1,2-dihydro-indol-3-ylidenemethyl)phenyl Ester.

M.p.: 238-240° C. ¹H NMR (DMSO-d₆+TFA): δ 1.3 (9H, s), δ 3.59 (3H, s), δ 3.81 (3H, s), δ 6.51 (1H, s), δ 7.15 (1H, s), δ 7.23 (2H, d, J=9 Hz), δ 7.42 (1H, s), δ 7.68 (1H, s), δ 7.73 (2H, d, J=9 Hz). HPLC purity: 96%. Elemental analysis (C₂₂H₂₄N₂O₅): found C (66.10%), H, (6.11%), N, (7.00%)—calculated C (66.65%), H, (6.10%), N, (7.07%).

A.3 n-Pentyl Carbamic Acid, (E)-4-(5,6-dimethoxy-2-oxo-1,2-dihydro-indol-3-ylidenemethyl)phenyl Ester.

M.p.: 173-175° C. ¹H NMR (DMSO-d₆): δ 0.9 (3H, t), δ 1.31 (2H, m), δ 1.5 (2H, quint.), δ 3.09 (2H, q), δ 3.58 (3H, s), δ 3.8 (3H, s), δ 6.51 (1H, s), δ 7.15 (1H, s), δ 7.28 (2H, d, J=9 Hz), δ 7.42 (1H, s), δ 7.70 (2H, d, J=9 Hz), δ 7.83 (1H, s), δ 10.33 (1H, s). HPLC purity: 97.1%. Elemental analysis (C₂₃H₂₆N₂O₅): found C (67.21%), H, (6.60%), N, (6.74%)—calculated C (67.30%), H, (6.38%), N, (6.82%)

A.4 n-Hexyl Carbamic Acid, (E)-4-(5,6-dimethoxy-2-oxo-1,2-dihydro-indol-3-ylidenemethyl)phenyl Ester.

M.p.: 168-170° C. ¹H NMR (DMSO-d₆+TFA): δ 0.9 (3H, t), δ 1.15-1.39 (6H, bb), δ 1.5 (2H, quint.), δ 3.07 (2H, q), δ 5.59 (3H, s), δ 5.8 (3H, s), 6.51 (1H, s), δ 7.1 (1H, s), δ 7.25 (2H, d, J=9 Hz), δ 7.42 (1H, s), δ 7.73 (2H, d, J=9 Hz), δ 7.82 (1H, t). HPLC purity: 96.8%. Elemental analysis (C₂₄H₂₈N₂O₅): found C (66.76%), H, (6.40%), N, (6.52%)—calculated C (67.91%), H, (6.65%), N, (6.60%).

A.5 4-Methoxyphenyl-carbamic Acid, (E)-4-(5,6-dimethoxy-2-oxo-1,2-dihydro-indol-3-ylidenemethyl)phenyl Ester.

M.p.: 189-191° C. ¹H NMR (DMSO d₆+TFA): δ 3.59 (3H, s), δ 3.71 (3H, s), δ 3.80 (3H, s), δ 6.53 (1H, s), δ 6.9 (2H, d), δ 7.13 (1H, s), δ 7.39 (2H, d, J=9 Hz), δ 7.41 (2H, d, J=9 Hz), δ 7.48 (1H, s, J=9 Hz), δ 7.75 (2H, d, J=9 Hz). HPLC purity: 93%. Elemental analysis (C₂₅H₂₂N₂O₆): found C (64.04%), H, (5.49%), N, (6.93%)—calculated C (67.26%), H, (4.97%), N, (6.27%).

METHOD II

B.1 Synthesis of (E)-4-methyl-piperazine-1-carboxylic Acid, 4-(5,6-dimethoxy-2-oxo-1,2-dihydro-indol-3-ylidenemethyl)phenyl Ester.

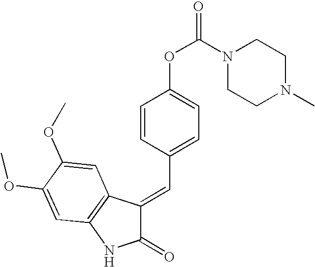

A solution of (E)-4-(5,6-dimethoxy-2-oxo-1,2-dihydro-indol-3-ylidenemethyl)phenyl-4-nitrophenylcarbonate of Example H.5 (0.2 g, 0.432 mmoles) in 8 ml of DMF, kept under stirring at room temperature, was dropwise added with 0.048 ml of N-methyl piperazine (0.432 mmoles). After 2 h the solvent was evaporated off and the resulting residue was purified by silica gel chromatography (eluent: dichloromethane/methanol 1/9), to give 0.11 g (0.259 mmoles, yield 60%) of a red-orange solid.

M.p.: 198-200° C. ¹H NMR (DMSO-d₆+AcOH-d₄) N-3070: δ 2.25 (3H, s), δ 2.4 (4H, m), δ 3.42 (2H, m), δ 3.58 (3H, s), δ 3.62 (2H, m), δ 3.79 (3H, s), δ 6.51 (1H, s), δ 7.12 (1H, s), δ 7.30 (2H, d, J=9 Hz), δ 7.43 (1H, s), δ 7.73 (2H, d, J=9 Hz). HPLC purity: 92.7%. Elemental analysis (C₂₃H₂₅N₃O₅): found C (64.02%), H, (5.98%), N, (9.71%)—calculated C (65.24%), H, (5.95%), N, (9.92%).

According to the procedure of Example B.1 (method II), the following compounds. were prepared (Table 3):

TABLE 3

| | | |
|---|---|---|
| B.2 | Morpholine-4-carboxylic acid, (E)-4-(5,6-dimethoxy-2-oxo-1,2-di- hydro-indol-3-yl- idenemethyl)phenyl ester | 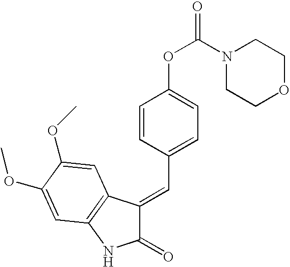 |
| B.3 | 4-(2-Hydroxyethyl)piperazine-1-car- boxylic acid, (E)-4-(5,6-di- methoxy-2-oxo-1,2-dihydro-in- dol-3-ylidenemethyl)phenyl ester | 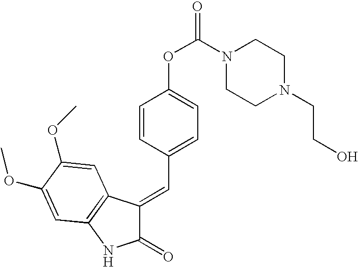 |
| B.4 | bis-(2-Hydroxyethyl)carbamic acid, (E)-4-(5,6-dimethoxy-2-oxo-1,2-di- hydro-indol-3-yl- idenemethyl)phenyl ester | 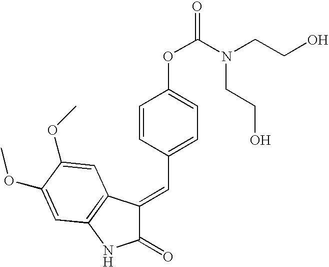 |
| B.5 | 4-(1′-Piperidino)-1-pipe- ridinecarboxylic acid, (E)-4-(5,6-di- methoxy-2-oxo-1,2-di- hydro-indol-3-yl- idenemethyl)phenyl ester | 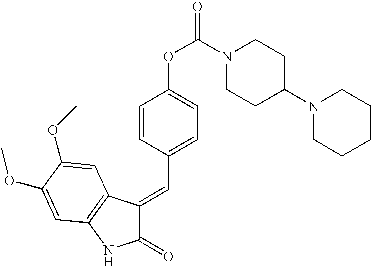 |
| B.6 | 4-[2-(2-Hydroxy- ethoxy)ethyl]piperazine-1-car- boxylic acid, (E)-4-(5,6-di- methoxy-2-oxo-1,2-dihydro-in- dol-3-ylidenemethyl)phenyl ester | 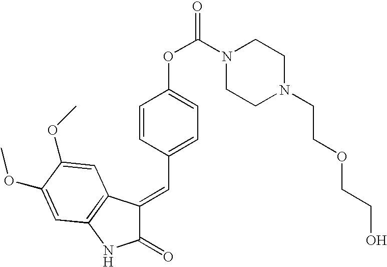 |
| B.7 | 4-Phenylpiperazine-1-carboxylic acid, (E)-4-(5,6-dimethoxy-2-oxo-1,2-di- hydro-indol-3-yl- idenemethyl)phenyl ester | 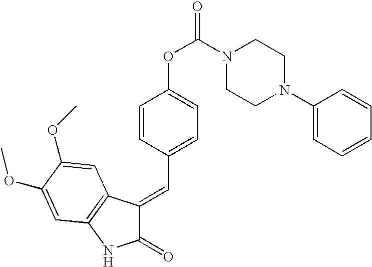 |

B.2 Morpholine-4-carboxylic Acid, (E)-4-(5,6-dimethoxy-2-oxo-1,2-dihydro-indol-3-ylidenemethyl)phenyl Ester.

M.p.: 255-257° C. ¹H NMR (DMSO-d₆-AcOH-d₄): δ 3.45 (2H, m), δ 3.59 (3H, s), δ 3.62 (2H, m), δ 3.7 (4H, m), δ 3.8 (3H, s), δ 6.51 (1H, s), δ 7.11 (1H, s), δ 7.30 (2H, d, J=9 Hz), δ 7.43 (1H, s), δ 7.75 (2H, d, J=9 Hz). HPLC purity: 92.7%. Elemental analysis (C₂₂H₂₂N₂O₆): found C (62.00%), H (5.38%), N, (6.45%)—calculated C (64.38%), H, (5.40%), N, (6.83%).

B.3 4-(2-Hydroxyethyl)piperazine-1-carboxylic Acid, (E)-4-(5,6-dimethoxy-2-oxo-1,2-dihydro-indol-3-ylidenemethyl)phenyl Ester.

M.p.: 217-220° C. ¹H NMR (DMSO-d₆+AcOH-d₄): δ 2.38-2.61 (6H, m), δ 3.48 (2H, m), δ 3.57 (2H, t), δ 3.61 (3H, s), δ 3.65 (2H, m), δ 3.81 (3H, s), δ 6.51 (1H, s), δ 7.11 (1H, s), δ 7.30 (2H, d, J=9 Hz), δ 7.43 (1H, s), δ 7.75 (2H, d, J=9 Hz). HPLC purity: 95.8%. Elemental analysis (C₂₄H₂₇N₃O₆): found C (62.83%), H, (6.13%), N, (9.35%)—calculated C (63.57%), H, (6.00%), N, (9.27%).

B.4 bis-(2-Hydroxyethyl)carbamic Acid, (E)-4-(5,6-dimethoxy-2-oxo-1,2-dihydro-indol-3-ylidenemethyl)phenyl Ester.

M.p.: 183-185° C. ¹H NMR (DMSO-d₆): δ 3.31 (2H, t), δ 3.39 (2H, t), δ 3.5 (2H, q), δ 3.56 (3H, s), δ 3.64 (2H, q), δ 3.81 (3H, s), δ 6.51 (1H, s), δ 7.11 (1H, s), δ 7.28 (2H, d, J=9 Hz), δ 7.43 (1H, s), δ 7.75 (2H, d, J=9 Hz), δ 10.33 (1H, s). HPLC purity: 94.43%. Elemental analysis (C22H24N2O7): found C (61.54%), H, (5.69%), N, (6.50)—calculated C (61.68%), H, (5.65%), N, (6.54%).

B.5 4-(1′-Piperidinyl)-1-piperidinecarboxylic Acid, (E)-4-(5,6-dimethoxy-2-oxo-1,2-dihydro-indol-3-ylidenemethyl)phenyl Ester.

M.p.: 235-237° C. ¹H NMR (DMSO-d₆+AcOH-d₄): δ 1.45-1.61 (8H, m), δ 1.88-1.92 (2H, m), δ 2.58-2.75 (6H, m), δ 2.86-3.04 (2H, m), δ 3.51 (3H, s), δ 3.79 (3H, s), δ 6.51 (1H, s), δ 7.12 (1H, s), δ 7.25 (2H, d, J=9 Hz), 7.42 (1H, s), δ 7.73 (2H, d, J=9 Hz). HPLC purity: 93.6%. Elemental analysis (C₂₈H₃₃N₃O₅): found C (66.86%), H, (6.71%), N, (8.87%)—calculated C (68.41%), H, (6.77%), N, (8.55%).

B.6 4-[2-(2-Hydroxyethoxy)ethyl]piperazine-1-carboxylic Acid (E)-4-(5,6-dimethoxy-2-oxo-1,2-dihydro-indol-3-ylidenemethyl)phenyl Ester.

M.p.: 170-172° C. ¹H NMR (DMSO-d₆+AcOH-d₄): δ 2.55 (2H, t), δ 3.17-3.68 (16H, m), δ 3.57 (3H, s), δ 3.78 (3H, s), δ 6.51 (1H, s), δ 7.12 (1H, s), δ 7.28 (2H, d, J=9 Hz), δ 7.42 (1H, s), δ 7.72 (2H, d, J=9 Hz). HPLC purity: 93.34%. Elemental analysis (C₂₇H₃₂N₂O₇): calculated C (62.77%), H, (6.28%), N, (8.45%)—found C (62.28%), H, (6.27%), N, (8.38%).

B.7 4-Phenylpiperazine-1-carboxylic Acid, (E)-4-(5,6-dimethoxy-2-oxo-1,2-dihydro-indol-3-ylidenemethyl)phenyl Ester.

M.p.: >260° C. ¹H NMR (DMSO-d₆+AcOH-d₄): δ 3.23 (4H, m), δ 3.58 (3H, s), δ 3.61 (2H, m), δ 3.76 (2H, m), δ 3.8 (3H, s), δ 6.51 (1H, s), δ 6.83 (1H, t), δ 6.99 (2H, d, J=9 Hz), δ 7.13 (1H, s), δ 7.25 (2H, d, J=9 Hz), δ 7.27 (1H, s), δ 7.32 (2H, d, J=9 Hz), δ 7.43 (1H, s), δ 7.75 (2H, d, J=9 Hz). HPLC purity: 96.2%. Elemental analysis (C₂₈H₂₇N₃O₅): found C (68.13%), H, (5.68%), N, (8.81%)—calculated C (69.26%), H, (5.61%), N, (8.65%).

### Example C.1

### Synthesis of (E)-4-(5,6-dimethoxy-2-oxo-1,2-dihydro-indol-3-ylidenemethyl)phenyl Phosphoric Acid

Step 1: Dibenzyl, (E)-4-(5,6-dimethoxy-2-oxo-1,2-dihydro-indol-3-ylidenemethyl)phenyl phosphate.

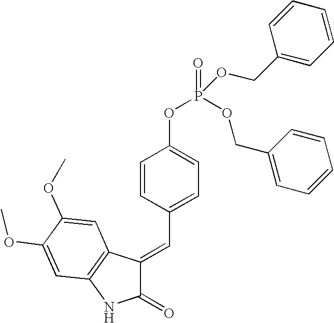

A solution of (E)-3-(4-hydroxybenzylidene)-5,6-dimethoxy-1,3-dihydro-indol-2-one (0.2 g, 0.67 mmole), CCl₄ (0.7 ml, 6.7 mmole) and DMAP (8 mg, 0.1%ₘ) in CH₃CN (15 ml) cooled at 0° C., was dropwise added with dibenzyl phosphite (DBP) (0.44 ml, 2.01 mmole) and N,N-diisopropylethylamine (DIEA) (0.46 ml, 2.68 mmole). The reaction mixture was stirred for 2 h, then the separated solid was recovered by filtration, washed with CH₃CN (5 ml) and dried to afford a yellow-orange solid (0.36 g, 96.4% yield).

M.p.: 162-164° C. ¹H NMR (DMSO-d₆): δ 3.52 (3H, s), δ 3.8 (3H, s), δ 5.18 (2H, s), δ 5.21 (2H, s), δ 6.51 (1H, s), δ 7.11 (1H, s), δ 7.35 (2H, d, J=9 Hz), δ 7.40 (10H, m), δ 7.51 (1H, s), δ 7.72 (2H, d, J=9 Hz), δ 10.33 (1H, s). HPLC purity: 96.3%. Elemental analysis (C₃₁H₂₈NO₃P): found C (64.78%), H, (5.06%), N, (2.51%)—calculated C (64.88%), H, (5.10%), N, (2.60%).

Step 2: (E)-4-(5,6-dimethoxy-2-oxo-1,2-dihydro-indol-3-ylidenemethyl)phenyl Phosphate, Disodium Salt.

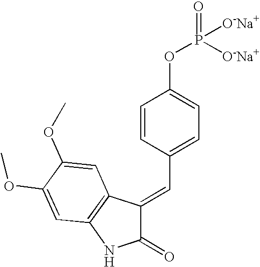

A solution of dibenzyl, (E)-4-(5,6-dimethoxy-2-oxo- 1,2-dihydro-indol-3-ylidenemethyl)phenyl phosphate from step 1 (0.2 g, 0.358 mmol) and NaI (0.215 g, 1.434 mmol) in CH₃CN (8 ml), was dropwise added with TMSCl (0.18 ml, 1.435 mmol). The reaction mixture was stirred at room temperature overnight. The solid precipitate was recovered by filtration, washed with CH₃CN (2 ml) and then resuspended under stirring for 3 h in n-hexane (3 ml). The product was filtered and dried to give a dark red solid (0.148 g, yield 98.2%).

M.p.: 209-211° C. ¹H NMR (DMSO-d₆) δ: 3.52 (3H, s), δ 3.8 (3H, s), δ 6.51 (1H, s), δ 7.11 (1H, s), δ 7.35 (2H, d, J=9 Hz), δ 7.51 (1H, s), δ 7.72 (2H, d, J=9 Hz), δ 10.33 (1H, s). HPLC purity: 93.6%.

Step 3: (E)-4-(5,6-dimethoxy-2-oxo-1,2-dihydro-indol-3-ylidenemethyl) Phenyl Phosphoric Acid.

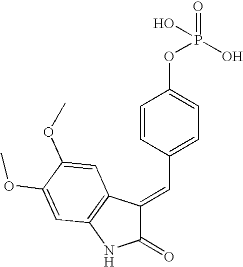

A solution of (E)-4-(5,6-dimethoxy-2-oxo-1,2-dihydro-indol-3-ylidenemethyl)phenyl phosphate, disodium salt from step 2 (0.350 g) in H₂O (2.5 ml) and 2N NaOH (1.8 ml) was acidified with 37% HCl to pH=0.07.

After cooling to 0-5° C., the solid precipitate was recovered by filtration and dried to afford the final product as a red powder (0.12 g, yield 38%).

M.p.: 218-220° C. ¹H NMR (DMSO-d₆): δ 3.52 (3H, s), δ 3.8 (3H, s), δ 6.51 (1H, s), δ 7.13 (1H, s), δ 7.35 (2H, d, J=9 Hz), δ 7.51 (1H, s), δ 7.70 (2H, d, J=9 Hz), δ 10.33 (1H, s). HPLC purity: 95.85%. Elemental analysis. (C₁₇H₁₆NO₇P): found C (52.02%), H, (4.19%), N, (3.34%)—calculated C (54.12%), H, (4.27%), N, (3.71%).

(E)-4-(5,6-Dimethoxy-2-oxo-1,2-dihydro-indol-3-ylidenemethyl)phenyl phosphoric acid, described in Example C.1, was also prepared according to the alternative method described in Example C.2.

### Example C.2

### Synthesis of (E)-4-(5,6-dimethoxy-2-oxo-1,2-dihydro-indol-3-ylidenemethyl)phenyl Phosphoric Acid

Step 1: (E)-4-(5,6-dimethoxy-2-oxo-1,2-dihydro-indol-3-ylidenemethyl)phenyl Diethyl Phosphate.

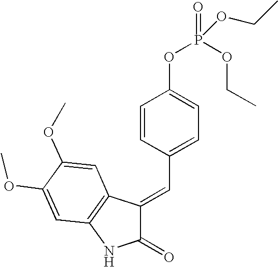

A suspension of (E)-3-(4-hydroxybenzylidene)-5,6-dimethoxy-1,3-dihydro-indol-2-one (310 mg, 1.043 mmoles) and TEA (0.22 ml, 1.095 mmoles) in 10 ml of dichloromethane, kept under stirring at −10° C., was dropwise added with a solution of diethyl chlorophosphate (0.23 ml, 1.095 mmoles) in 2 ml of dichloromethane. After completion of the addition, temperature was allowed to raise to room temperature and stirring was maintained for 12 h. Solvent was evaporated off, the resulting residue was dissolved in 150 ml of AcOEt and added with a 10% NaCl aqueous solution to precipitate a solid which was filtered and, subsequently, left under stirring with 50 ml of water. Upon filtration and drying, 255 mg of an orange-red solid were obtained (0.59 mmoles, yield 56%).

M.p.: 139-141° C. ¹H NMR (DMSO d₆): δ 1.29 (6H, t), δ 3.60 (3H, s), δ 3.81 (3H, s), δ 4.19 (4H, q), δ 6.51 (1H, s), δ 7.09 (1H, s), δ 7.39 (2H, d, J=9 Hz), δ 7.41 (1H, s), δ 7.78 (2H, d, J=9 Hz), δ 10.33 (1H, s). HPLC purity: 95%. Elemental analysis. (C₂₁H₂₄NO₇P): found C (55.91%), H, (5.29%), N, (3.14%), P (8.74%)—calculated C (58.20%), H, (5.58%), N, (3.23%), P (7.15%).

Step 2: (E)-4-(5,6-Dimethoxy-2-oxo-1,2-dihydro-indol-3-ylidenemethyl)phenyl Phosphoric Acid.

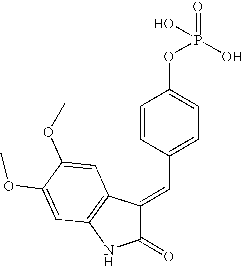

A solution of (E)-4-(5,6-dimethoxy-2-oxo-1,2-dihydro-indol-3-ylidenemethyl)phenyl diethyl phosphate (4.4 g, 10 mmoles, 1 eq.) from step 1 in dry dichloromethane (110 mL) was cooled to 0°-5° C., then dropwise added with trimethylsilyl bromide(7.9 mL, 6 mmoles, 6 eq.). The resulting reaction mixture was kept under stirring at room temperature for 24 hours. After completion of the reaction, the solvent was evaporated off under reduced pressure and the crude was suspended in n-hexane (180 mL) and 2N HCl in ethyl ether (0.96 mL, 2 eq.). The suspension was filtered to obtain a dark red solid, which was suspended in ethyl ether (40 mL) and water (0.11 mL), then filtered to give the desired product (2.7 g), as an isomeric mixture E/Z (85%-15%), ¹H NMR (DMSO d₆): δ 3.50 (3H, s), δ 3.8 (3H, s), δ 6.5 (1H, s), δ 7.1 (1H, s), δ 7.3 (2H, d); δ 7.39 (1H, s), δ 7.7 (2H, d), δ 10.3 (1H, s), signals isomer: δ 3.79 (s); δ 6.45 (s); δ 7.2 (d); δ 7.38 (s); δ 7.6 (s). elemental analysis. (C₂₁H₂₄NO₇P): found C (51.83%), H, (4.37%), N, (3.55%),—calculated C (54.12%), H, (3.55%), N, (3.71%). Melting point 218° C.-220° C.

D.1 Synthesis of (E) 3-[4-(2-hydroxyetoxy)benzylidene]-5,6-dimethoxy-1,3-dihydro-indol-2-one.

Step 1: 4-(2-hydroxyethoxy)-benzaldehyde.

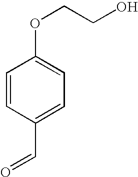

A solution of p-hydroxybenzaldehyde (2 g, 0.016 moles) and K₂CO₃ (2.72 g, 0.0197 moles) in DMA (10 ml), kept under strong stirring and at room temperature, was dropwise added with 2-bromoethanol (1.39 ml, 0.0197 moles). After 3 days, the reaction was quenched by addition of 200 ml of H₂O and the mixture was extracted with 3×50 ml of Et₂O and 2×50 ml of AcOEt. The combined organic phases were dried over Na₂SO₄ and the solvent was evaporated off to afford an oil which was purified by filtration on silica gel (eluent first with i-Pr₂O then with Et₂O) thereby obtaining 1.6 g (0.0096 moles, yield 60%) of a colorless oil.

¹H NMR (DMSO-d₆) N-3425: δ 3.74 (2H, q), δ 4.11 (2H, t), δ 4.92 (1H, t), δ 7.13 (2H, dd, J=9.5 Hz), δ 7.86 (2H, dd, J=9.5 Hz), δ 9.87 (1H, s).

Step 2: (E) 3-[4-(2-hydroxyetoxy)benzylidene]-5,6-dimethoxy-1,3-dihydro-indol-2-one.

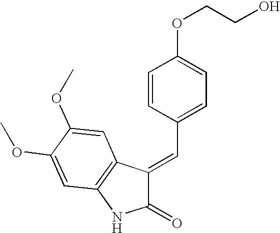

Two drops of piperidine were dropped into a solution of 1,3-dihydro-5,6-dimethoxy-(1H)-indol-2-one (0.3 g, 0.0016 moles) and 4-(2-hydroxyethoxy)-benzaldehyde (0.31 g, 0.0019 moles) in 5 ml of EtOH, kept under stirring and at 60° C., and subsequently the mixture was refluxed. After 4 h the solution was cooled at room temperature and the formed precipitate was filtered, recrystallized from 2 ml of AcOEt and filtered to afford 300 mg (0.000878 moles, yield 54.8%) of a brown solid.

M.p.:=199-202° C. ¹H NMR (DMSO-d₆) N-3425: δ 3.61 (3H, s), δ 3.74 (2H, q), δ 3.78 (3H, s), δ 4.07 (2H, t), δ4.89 (1H, t), δ 6.51 (1H, s), δ 7.09 (2H, d), δ 7.24 (1H, s), δ 7.39 (1H, s), δ 7.68 (2H, d), δ 10.29 (1H, s). HPLC purity: 92.1%. Elemental analysis. (C₁₉H₁₉NO₅): found C (65.64%), H, (5.75%), N, (3.98%)—calculated C (66.85%), H, (5.61%), N, (4.10%).

D.2 Synthesis of tert-butyl (E)-[4-(5,6-dimethoxy-2-oxo-1,2-dihydro-indol-3-ylidenemethyl)phenoxy]acetate.

Step 1: 4-[(tert-butoxycarbonylmethyl)oxy]benzaldehyde.

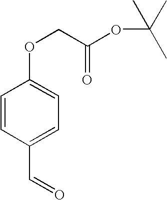

A solution of 4-hydroxybenzaldehyde (2 g, 0.0164 mole) and TEA (6.8 ml, 0.0492 mole) in AcOEt (50 ml), at room temperature, was dropwise added with tert-butyl-bromoacetate (5.8 ml, 0.0394 mole). The mixture was stirred 3 days, then TEA*HCl was precipitated off and filtration waters were extracted with a 10% Na₂CO₃ aqueous solution (30 ml). The organic phase was separated, washed with a 10% Na₂CO₃ aqueous solution (2×30 ml) and dried over dry Na₂SO₄. The solvent was evaporated off under reduced pressure, the residue was purified by column chromatography with silica gel, eluting with a dichloromethane/isopropyl ether 95/5 mixture, thereby obtaining a waxy white solid (0.142 g, 37% yield).

¹H NMR (DMSO-d₆) N-3425: δ 1.43 (9H, s), δ 4.18 (2H, s), δ 7.05 (2H, d, J=8.72 Hz), δ 7.8 (2H, d, J=8.72 Hz), δ 9.87 (1H, s).

Step 2: Synthesis of tert-butyl (E)-[4-(5,6-dimethoxy-2-oxo-1,2-dihydro-indol-3-ylidenemethyl)phenoxy]acetate.

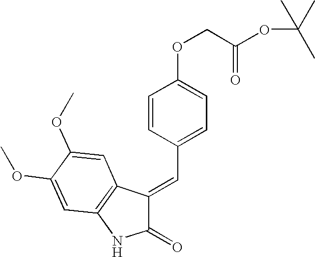

A solution of 4-[(tert-butoxycarbonylmethyl)oxy] benzaldehyde from Step 1 (1.027 g, 4.3 mmole), 5,6-dimethoxy-1,3-dihydro-indol-2-one (0.7 g, 3.6 mmole) and piperidine (0.4 ml, 4 mmole) in DMSO (7 ml) was stirred for 48 h at room temperature. The reaction mixture was then poured into H₂O (100 ml) and extracted with AcOEt (6×50 ml). The organic phase was dried over sodium sulfate anhydrous, filtered and the solvent was distilled off under reduced pressure. The residue was purified by chromatography on a silica gel column, eluting with an AcOEt/n-hexane 8/2 mixture. The purified product was recrystallized from AcOEt (5 ml) to afford the desired product as a red solid (0.16 g, yield 11%).

M.p.: 158-160° C. ¹H NMR (DMSO-d₆): δ 1.41 (9H, s), δ 3.55 (3H, s), δ 3.78 (3H, s), δ 4.72 (2H, s), δ 6.51 (1H, s), δ 7.08 (2H, d, J=9 Hz), δ 7.21 (1H, s), δ 7.39 (1H, s), δ 7.68 (2H, d, J=9 Hz), δ 10.31 (1H, s). HPLC purity: 95.62%. Elemental analysis: found C (67.03%), H, (6.06%), N, (3.52%)—calculated C (67.14%), H, (6.12%), N, (3.40%). (C₂₃H₂₅NO₆).

D.3 Synthesis of (E) [4-(5,6-dimethoxy-2-oxo-1,2-dihydro-indol-3-ylidenemethyl)phenoxy]acetic Acid.

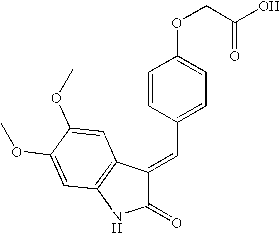

A solution of (E)-[4-(5,6-dimethoxy-2-oxo-1,2-dihydro-indol-3-ylidenemethyl)phenoxy]acetate tert-butyl ester (0.05 g; 0.1 mmoles) in 1,4-dioxane/hydrochloric acid (4.0 M; 5 mL) was kept under stirring at room temperature for 22 h. The precipitated solid was collected and washed with CH₂Cl₂, to afford 0.035 g (yield: 81%) of a red solid consisting, according to ¹H NMR analysis, of the E isomer of the carboxylic acid (94%), Z isomer of the carboxylic acid (5%), unreacted ester (1%, non detectable by TLC). M.p.: 220-223° C.

¹H NMR (DMSO-d₆): δ 13.1 (1H, br, COOH); 10.29 (1H, s, NH); 7.67 (2H, d, J=8.3 Hz, ArH-3); 7.38 (1H, s, ═CH); 7.19 (1H, s, H-4′); 7.07 (2H, d, J=8.3 Hz, ArH-2); 6.50 (1H, s, H-7′); 4.76 (2H, s, OCH₂); 3.77 (3H, s, OCH₃); 3.59 (3H, s, OCH₃). HPLC purity: 97.32%. Elemental analysis: found C (67.03%), H, (6.06%), N, (3.52%)—calculated C (67.14%), H, (6.12%), N, (3.40%). (C₂₃H₂₅NO₆).

### Example E.1

### Synthesis of Benzoic Acid, (E)-4-(5,6-dimethoxy-2-oxo-1,2-dihydro-indol-3-ylidenemethyl)phenyl Ester

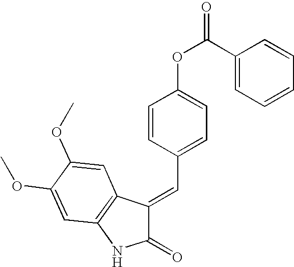

A suspension of (E)-3-(4-hydroxybenzylidene)-5,6-dimethoxy-1,3-dihydro-indol-2-one (266 mg, 0.895 mmoles), and TEA (0.175 ml, 0.94 mmoles) in 3 ml of DCM, kept under stirring at −5° C., was dropwise added with a solution of benzoyl chloride (0.146 ml, 0.94 mmoles) in 1 ml of DCM.

After 24 h the solvent was evaporated off and the resulting crude was treated with 4 ml of water, filtered and dried. The solid was recrystallized from 4 ml of AcOEt to afford 311 mg of product (0.774 mmoles, yield 97%).

M.p.: 228-230° C. ¹H NMR (DMSO-d₆): δ 3.58 (3H, s), δ 3.81 (3H, s), δ 6.51 (1H, s), δ 7.11 (1H, s), δ 7.48 (1H, s), δ 7.51 (2H, d), 6 7.67 (2H, t)δ 7.79 (1H, d), δ 7.81 (2H, d), δ 8.18 (2H, d), δ 10.33 (1H, s). HPLC purity: 98.5%. Elemental analysis (C₂₄H₁₉NO₅): found C (70.81%), H, (4.82%), N, (3.5 1%)—calculated C (71.81%), H, (4.77%), N, (3.49%).

According to the procedure of Example E.1 the following compounds were prepared (Table 4):

TABLE 4

| | | |
|---|---|---|
| E.2 | Acetic acid, (E)-4-(5,6-dimethoxy-2-oxo-1,2-di- hydro-indol-3-ylidenemethyl)phenyl ester | 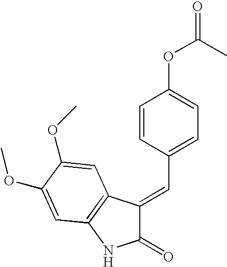 |
| E.3 | Nicotinic acid, (E)-4-(5,6-dimethoxy-2-oxo-1,2-di- hydro-indol-3-yl- idenemethyl)phenyl ester | 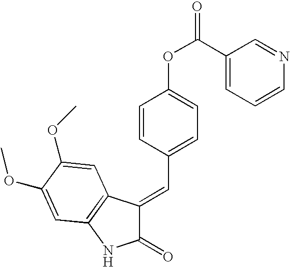 |
| E.4 | Succinic acid, (E)-4-(5,6-dimethoxy-2-oxo-1,2-di- hydro-indol-3-ylidenemethyl)phenyl ester, ethyl ester. | 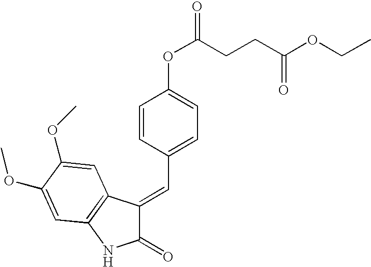 |
| E.5 | Succinic acid, (E)-4-(5,6-dimethoxy-2-oxo-1,2-di- hydro-indol-3-ylidenemethyl)phenyl ester, benzyl ester. | 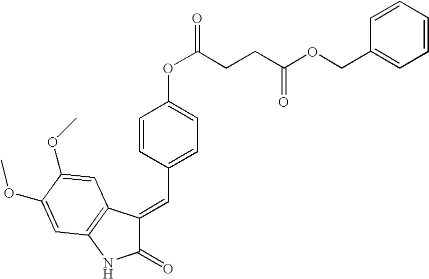 |

E.2 Acetic Acid, (E)-4-(5,6-dimethoxy-2-oxo-1,2-dihydro-indol-3-ylidenemethyl)phenyl Ester.

M.p.: 189-191° C. ¹H NMR (DMSO-d₆): δ 2.31 (3H, s), δ 3.58 (3H, s), δ 3.81 (3H, s), δ 6.51 (1H, s), δ 7.09 (1H, s), δ 7.29 (2H, d), δ 7.33 (1H, s), δ 7.78 (2H, d), δ 10.33 (1H, s). HPLC purity: 94.9%. Elemental analysis (C₁₉H₁₇NO₅): found C (66.27%), H, (4.94%), N, (4.04%)—calculated C (67.25%), H, (5.05%), N, (4.13%).

E.3 Nicotinic Acid, (E)-4-(5,6-dimethoxy-2-oxo-1,2-dihydro-indol-3-ylidenemethyl)phenyl Ester.

M.p.: 227-229° C. ¹H NMR (DMSO-d₆): δ 3.58 (3H, s), δ 3.81 (3H, s), δ 6.51 (1H, s), δ 7.11 (1H, s), δ 7.48 (1H, s), δ 7.51 (2H, d, J=8.60 Hz), δ 7.69 (1H, dd, J=7.87 Hz, J=4.76 Hz), δ 7.85 (2H, d, J=8.60 Hz), δ 8.51 (1H, d, J=7.87), δ 8.92 (1H, dd, J=1.65 Hz, J=4.76 Hz), δ 9.29 (1H d, J=1.65 Hz), δ 10.33 (1H, s). HPLC purity: 95.6%. Elemental analysis (C₂₃H₁₈N₂O₅): found C (66.93%), H, (4.41%), N, (6.82%)—calculated C (68.65%), H, (4.51%), N, (6.96%).

E.4 Succinic Acid, (E)-4-(5,6-dimethoxy-2-oxo-1,2-dihydro-indol-3-ylidenemethyl)phenyl Ester, Ethyl Ester.

¹H NMR (DMSO-d₆): δ 1.2 (t, 3H), δ 2.7 (2H, t), δ 2.87 (t, 2H), δ 3.58 (3H, s), δ 3.81 (3H, s), δ 4.15 (q, 2H), δ 6.51 (1H, s), δ 7.11 (1H, s), δ 7.28 (2H, d), δ 7.45 (1H, s), δ 7.77 (2H, d), δ 10.35 (1H, s).

E.5 Succinic Acid, (E)-4-(5,6-dimethoxy-2-oxo-1,2-dihydro-indol-3-ylidenemethyl)phenyl Ester, Benzyl Ester.

¹H NMR (DMSO-d₆): δ 2.77 (t, 2H), δ 2.9 (2H, t), δ 3.56 (s, 3H), δ 3.79 (3H, s), δ 5.15 (2H, s), δ 6.51 (s, 1H), δ 7.08 (1H, s), δ 7.21 (2H, d), δ 7.36-7.38 (5H, m), δ 7.43 (1H, s), δ 7.74 (2H, d), δ 10.35 (1H, s).

### Example F.1

### Synthesis of n-decanoic Acid, (E)-4-(5,6-dimethoxy-2-oxo-1,2-dihydro-indol-3-ylidenemethyl)phenyl Ester

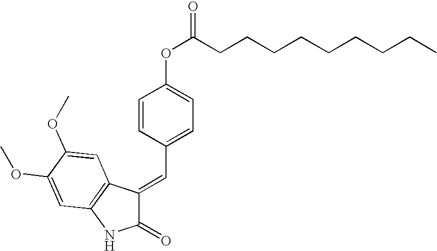

A solution of (E)-3-(4-hydroxybenzylidene)-5,6-dimethoxy-1,3-dihydro-indol-2-one (0.2 g, 1 eq.) and n-decanoic acid (0.174 g, 1.5 eq.) in 2 ml of dry DMF, kept under stirring at room temperature, was added with 4-dimethylaminopyridine (8 mg, 0.1 eq.) and 1-(3-dimethylpropyl)-3-ethylcarbodiimide hydrochloride (EDCl) (0.191 g, 1.5 eq.). After 12 h the reaction mixture was poured into 50 ml of H₂O and extracted with 30 ml of AcOEt. The organic phase was dried over Na₂SO₄, solvent was evaporated off and the residue was purified by silica gel chromatography (eluent AcOEt:n-hexane 6:4). 140 mg of a solid were recovered, which was recrystallized from AcOEt to afford n-decanoic acid, (E)-4-(5,6-dimethoxy-2-oxo-1,2-dihydro-indol-3-ylidenemethyl)phenyl ester as a red-orange solid (0.107 g; yield 35%).

M.p.: 138-140° C. ¹H NMR (DMSO-d₆): δ 0.87 (3H, t), δ 1.27-1.38 (12H, bb), δ 1.65 (2H, m), δ 2.6 (2H, t), δ 3.56 (3H, s), δ 3.79 (3H, s), δ 6.51 (1H, s), δ 7.08 (1H, s), δ 7.27 (2H, d, J=9 Hz), δ 7.43 (1H, s), δ 7.74 (2H, d, J=9 Hz), δ 10.35 (1H, s). HPLC purity: 98.1%. Elemental analysis (C₂₇H₃₃NO₅): found C (71.48%), H, (7.27%), N, (3.10%)—calculated C (71.82%), H, (7.37%), N, (3.10%).

According to the procedure of Example F.1 the compounds of the following table were prepared:

TABLE 5

| | | |
|---|---|---|
| F.2 | 5-tert-Butoxycarbonylamino pentanoic acid, (E)-4-(5,6-di- methoxy-2-oxo-1,2-dihydro-indol-3-yl- idenemethyl)phenyl ester | 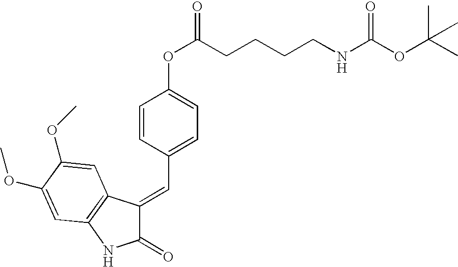 |
| F.3 | [2-(2-Methoxy-ethoxy)-eth- oxy]acetic acid, (E)-4-(5,6-di- methoxy-2-oxo-1,2-dihydro-indol-3-yl- idenemethyl)phenyl ester | 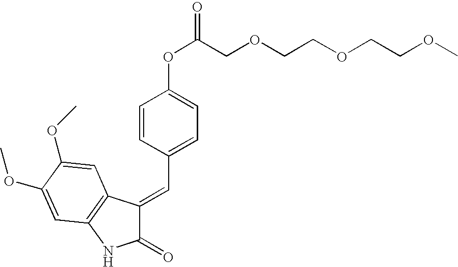 |
| F.4 | 1-Methyl-1H-pyrrole-2-carboxylic acid, (E)-4-(5,6-dimethoxy-2-oxo-1,2-di- hydro-indol-3-yl- idenemethyl)phenyl ester | 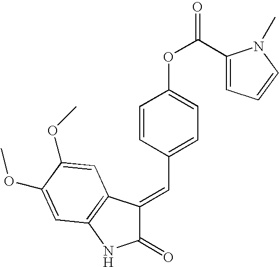 |
| F.5 | Pyrazine-2-carboxylic acid, (E)-4-(5,6-di- methoxy-2-oxo-1,2-dihydro-in- dol-3-ylidenemethyl)phenyl ester | 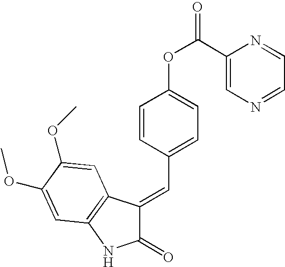 |
| F.6 | Pivalic acid, (E)-4-(5,6-dimethoxy-2-oxo-1,2-di- hydro-indol-3-yl- idenemethyl)phenyl ester | 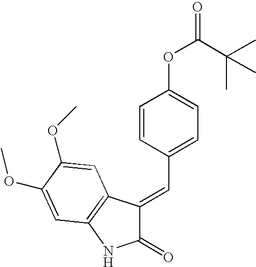 |
| F.7 | 1-Adamantyl carboxylic acid, (E)-4-(5,6-di- methoxy-2-oxo-1,2-dihydro-in- dol-3-ylidenemethyl)phenyl ester | 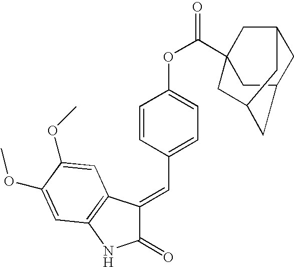 |
| F.8 | 2-(S)-tert-Butoxycarbonylamino propionic acid, (E)-4-(5,6-di- methoxy-2-oxo-1,2-dihydro-indol-3-yl- idenemethyl)phenyl ester | 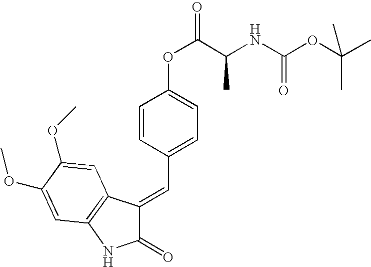 |
| F.9 | 4-Cyanobenzoic acid, (E)-4-(5,6-di- methoxy-2-oxo-1,2-dihydro-indol-3-yl- idenemethyl)phenyl ester | 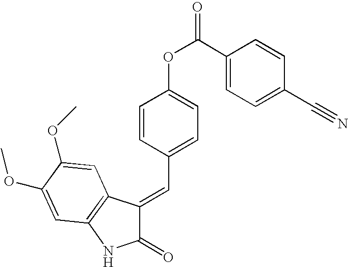 |
| F.10 | 4-(5-Tetrazolyl)benzoic acid, (E)-4-(5,6-di- methoxy-2-oxo-1,2-dihydro-in- dol-3-ylidenemethyl)phenyl ester | 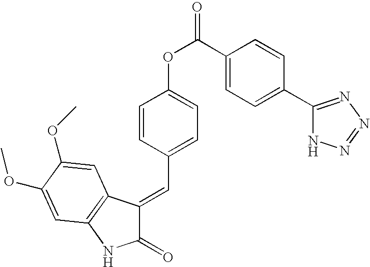 |
| F.11 | 5-Isoxazolelcarboxylic acid, (E)-4-(5,6-di- methoxy-2-oxo-1,2-dihydro-in- dol-3-ylidenemethyl)phenyl ester | 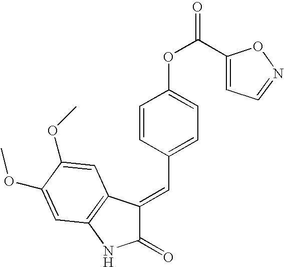 |
| F.12 | Acetylamino acetic acid, (E)-4-(5,6-di- methoxy-2-oxo-1,2-dihydro-indol-3-yl- idenemethyl)phenyl ester | 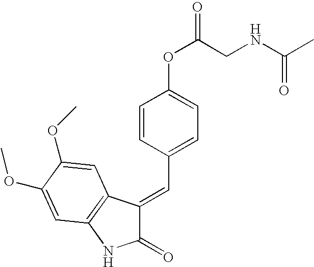 |
| F.13 | 2-Hydroxynicotinic acid, (E)-4-(5,6-di- methoxy-2-oxo-1,2-dihydro-indol-3-yl- idenemethyl)phenyl ester | 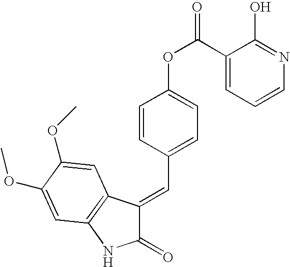 |

F.2. 5-tert-Butoxycarbonylamino-pentanoic Acid, (E)-4-(5,6-dimethoxy-2-oxo-1,2-dihydro-indol-3-ylidenemethyl)phenyl Ester.

M.p.: 101-103° C. ¹H NMR (DMSO-d₆): δ 1.38 (9H, s), δ 1.48 (2H, quint.), δ 1.64 (2H, quint.), δ 2.62 (2H, t), δ 2.96 (2H, q), δ 3.56 (3H, s), δ 3.79 (3H, s), δ 6.51 (1H, s), δ 6.84 (1H, t), δ 7.08 (1H, s), δ 7.27 (2H, d, J=9 Hz), δ 7.43 (1H, s), δ 7.74 (2H, d, J=9 Hz), δ 10.35 (1H, s). HPLC purity: 97.11%. Elemental analysis (C27H32N2O7): found C (64.85%), H, (6.49%), N, (5.51%)—calculated C (65.31%), H, (6.5%), N, (5.64%).

F.3 [2-(2-Methoxy-ethoxy)-ethoxy]acetic Acid, (E)-4-(5,6-dimethoxy-2-oxo-1,2-dihydro-indol-3-ylidenemethyl)phenyl Ester.

M.p.: 117-119° C. ¹H NMR (DMSO-d₆): δ 3.25 (3H, s), δ 3.45 (2H, t), δ 3.54 (2H, t), δ 3.56 (3H, s), δ 3.59 (2H, t), δ 3.71 (2H, t), δ 3.79 (3H, s), δ 4.46 (2H, s), δ 6.51 (1H, s), δ 7.08 (1H, s), δ 7.33 (2H, d, J=9 Hz), δ 7.44 (1H, d), δ 7.76 (2H, d, J=9 Hz), δ 10.36 (1H, s). HPLC purity: 92.9%. Elemental analysis (C₂₄H₂₇NO₉): found C (62.83%), H, (5.91%), N, (3.09%)—calculated C (63.01%), H, (5.95%), N, (3.06%).

F.4 1-Methyl-1H-pyrrole-2-carboxylic Acid, (E)-4-(5,6-dimethoxy-2-oxo-1,2-dihydro-indol-3-ylidenemethyl)phenyl Ester.

M.p.: 165-167° C. ¹H NMR (DMSO-d₆): δ 3.59 (3H, s), δ 3.79 (3H, s), δ 3.91 (3H, s), δ 6.22 (1H, dd, J=4.02 Hz, J=2.5), δ 6.52 (1H, s), δ 7.14 (1H, s), δ 7.15 (1H, d, J=2.5), δ 7.26 (1H, d, J=4.02), δ 7.38 (2H, d, J=9 Hz), δ 7.465 (1H, s), δ 7.78 (2H, d, J=9 Hz), δ 10.36 (1H, s). HPLC purity: 96.5%. Elemental analysis (C₂₃H₂₀N₂O₅): found C (67.23%), H (5.02%), N, (6.69%)—calculated C (68.31%), H, (4.98%), N, (6.93%).

F.5 Pyrazine-2-carboxylic Acid, (E)-4-(5,6-dimethoxy-2-oxo-1,2-dihydro-indol-3-ylidenemethyl)phenyl Ester.

M.p.: >250° C. ¹H NMR (DMSO-d₆): δ 3.58 (3H, s), δ 3.79 (3H, s), δ 6.52 (1H, s), δ 7.11 (1H, s), δ 7.48 (1H, s), δ 7.52 (2H, d, J=9 Hz), δ 7.83 (2H, d, J=9 Hz), δ 8.93 (1H, m), δ 9.10 (1H, d, J=2.3 Hz), δ 9.43 (1H, s), δ 10.37 (1H, s). HPLC purity: 91.3%. Elemental analysis (C₂₂H₁₇N₃O₅): found C (65.18%), H, (4.27%), N, (10.52%)—calculated C (65.5%), H, (4.25%), N, (10.42%).

F.6 Pivalic Acid, (E)-4-(5,6-dimethoxy-2-oxo-1,2-dihydro-indol-3-ylidenemethyl)phenyl Ester.

¹H NMR (DMSO-d₆): δ 1.33 (9H, s), δ 3.58 (3H, s), δ 3.80 (3H, s), δ 6.52 (1H, s), δ 7.1 (1H, s), δ 7.26 (2H, d, J=8.6 Hz), δ 7.43 (1H, s), δ 7.75 (2H, d, J=8.6 Hz), δ 10.36 (1H, s). HPLC purity: 99.3%. Elemental analysis (C₂₂H₂₃NO₆): found C (69.25%), H, (6.06%), N, (3.68%)—calculated C (69.28%), H, (6.08%), N, (3.67%).

F.7 1-Adamantyl Carboxylic Acid, (E)-4-(5,6-dimethoxy-2-oxo-1,2-dihydro-indol-3-ylidenemethyl)phenyl Ester.

¹H NMR (DMSO-d₆): δ 1.74 (6H, m), δ 2.013 (6H, m), δ 2.055 (3H, m), δ 3.57 (2H, s), δ 3.79 (3H, s), δ 6.51 (1H, s), δ 7.10 (1H, s), δ 7.24 (2H, d J=8.45 Hz), δ 7.43 (1H, s), δ 7.75 (2H, d, J=8.5 Hz), δ 10.35 (1H, s). HPLC purity: 97.8%. Elemental analysis (C₂₈H₂₉NO₆): found C (73.46%), H, (6.35%), N, (3.03%)—calculated C (73.18%), H, (6.36%), N, (3.05%).

F.8 2-(S)-tert-Butoxycarbonylamino propionic Acid, (E)-4-(5,6-dimethoxy-2-oxo-1,2-dihydro-indol-3-ylidenemethyl)phenyl Ester.

¹H NMR (DMSO-d₆): δ 3.56 (3H, s), δ 3.79 (3H, s), δ 4.24 (1H, q, J=7 Hz), δ 6.51 (1H, s), δ 7.07 (1H, s), δ 7.25 (2H, d, J=8.5 Hz), δ 7.43 (1H, s), δ 7.56 (1H, d, J=7 Hz), δ 7.77 (2H, d, J=8.5), δ 10.35 (1H, s). HPLC purity: 96.2%. Elemental analysis (C₂₅H₂₈N₂O₇): found C (64.04%), H, (5.92%), N, (6.02%)—calculated C (64.09%), H, (6.02%), N, (5.98%).

F.9 4-Cyanobenzoic Acid, (E)-4-(5,6-dimethoxy-2-oxo-1,2-dihydro-indol-3-ylidenemethyl)phenyl Ester.

¹H NMR (DMSO-d₆): δ 3.59 (3H, s), δ 3.80 (3H, s), δ 6.52 (1H, s), δ 7.12 (1H, s), δ 7.47 (1H, s), δ 7.51 (2H, d, J=8.59 Hz), δ 7.83 (2H, d, J=8.59 Hz), δ 8.11 (2H, d, J=8.34), δ 8.32 (2H, d, J=8.34), δ 10.37 (1H, s). HPLC purity: 93.7%. Elemental analysis (C₂₅H₁₈N₂O₅): found C (67.85%), H, (4.31%), N, (6.15%)—calculated C (70.42%), H, (4.25%), N, (6.57%).

F.10 4-(5-Tetrazolyl)benzoic Acid, (E)-4-(5,6-dimethoxy-2-oxo-1,2-dihydro-indol-3-ylidenemethyl)phenyl Ester.

¹H NMR (DMSO-d₆): δ 3.59 (3H, s), δ 3.80 (3H, s), δ 6.50 (1H, s), δ 7.13 (1H, s), δ 7.48 (1H, s), δ 7.51 (2H, d J=8.5 Hz), δ 7.83 (2H, d, J=8.5Hz), δ 8.28 (2H, d, J=8.4), δ 8.37 (2H, d, J=8.4), δ 10.37 (1H, s).

F.11 5-Isoxazolelcarboxylic Acid, (E)-4-(5,6-dimethoxy-2-oxo-1,2-dihydro-indol-3-ylidenemethyl)phenyl Ester.

¹H NMR (DMSO-d₆): δ 3.57 (3H, s), δ 3.79 (3H, s), δ 6.52 (1H, s), δ 7.07 (1H, s), δ 7.46 (1H, s), δ 7.51 (2H, d J=8.5 Hz), δ 7.56 (1H, d, J=1.5 Hz), δ 7.82 (2H, d, J=8.5), δ 8.97 (1H, d, J=1.5), δ 10.37 (1H, s). HPLC purity: 91.14%. Elemental analysis (C₂₁H₁₆N₂O₆): found C (64.87%), H, (4.26%), N, (7.20%)—calculated C (64.28%), H, (4.11%), N, (7.14%).

F.12 Acetylamino Acetic Acid, (E)-4-(5,6-dimethoxy-2-oxo-1,2-dihydro-indol-3-ylidenemethyl)phenyl Ester.

¹H NMR (DMSO-d₆): 1.95 (3H, s), 3.6 (3H, s), 3.79 (3H, s), 4.15 (2H, d), 6.50 (1H, s), 7.1 (1H, s), 7.25 (2H, d), 7.35 (1H, s), 7.8 (2H, d), 8.5 (1H, m), 10.38 (1H, s).

F.13 2-Hydroxynicotinic Acid, (E)-4-(5,6-dimethoxy-2-oxo-1,2-dihydro-indol-3-ylidenemethyl)phenyl Ester.

¹H NMR (DMSO-d₆): 3.58 (3H, s), 3.79 (3H, s), 6.37 (1H, t), 6.52 (1H, s), 7.13 (1H, s), 7.36 (2H, d), 7.45(1H, s), 7.77-7.79 (3H, m), 8.34-8.37 (1H, dd), 10.36 (1H, s), 12.28 (1H, s).

### Example G.1

### Synthesis of n-decanoic Acid, (Z)-4-(5,6-dimethoxy-2-oxo-1,2-dihydro-indol-3-ylidenemethyl)phenyl Ester

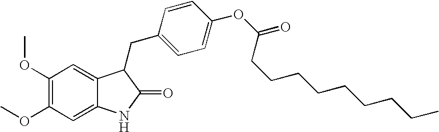

The product was isolated from the column chromatography of Example F.1. The fraction containing the product (0.05 g) was recrystallized from AcOEt (0.9 ml), to afford the product as a red solid (0.02 g, yield 7%).

M.p.: 95-97° C. ¹H NMR (DMSO-d₆): δ 0.87 (3H, t), δ 1.27-1.39 (12H, bb), δ 1.65 (2H, m), δ 2.59 (2H, t), δ 3.78 (6H, s), δ 6.46 (1H, s), δ 7.19 (2H,. d, J=8.5 Hz), δ 7.40 (1H, s), δ 7.64 (1H, s), δ 8.38 (2H, d, J=8.5 Hz), δ 10.36 (1H, s). HPLC purity: 98.1%. Elemental analysis (C₂₇H₃₃NO₅): found C (71.84%), H, (7.23%), N, (3.13%)—calculated C (71.82%), H, (7.37%), N, (3.10%).

According to the procedure of Example G.1 the following compounds were prepared (Table 6).

TABLE 6

| | | |
|---|---|---|
| G.2 | 5-tert-Butoxycarbonylamino-pen- tanoic acid, (Z)-4-(5,6-di- methoxy-2-oxo-1,2-dihydro-indol-3-yl- idenemethyl)phenyl ester | 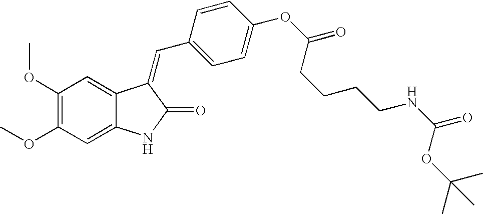 |
| G.3 | 1-Methyl-1H-pyrrole-2-carboxylic acid, (Z)-4-(5,6-dimethoxy-2-oxo-1,2-di- hydro-indol-3-yl- idenemethyl)phenyl ester | 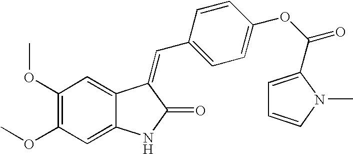 |
| G.4 | 1-Adamantyl carboxylic acid, (Z)-4-(5,6-di- methoxy-2-oxo-1,2-dihydro-in- dol-3-ylidenemethyl)phenyl ester | 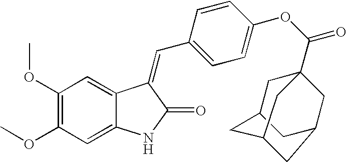 |
| G.5 | 2-(S)-tert-Butyoxycarbonylamino propionic acid, (Z)-4-(5,6-dimethoxy-2-oxo-1,2-di- hydro-indol-3-yl- idenemethyl)phenyl ester | 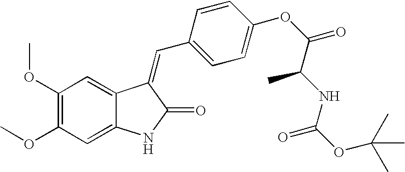 |
| G.6 | Pivalic acid, (Z)-4-(5,6-dimethoxy-2-oxo-1,2-di- hydro-indol-3-yl- idenemethyl)phenyl ester | 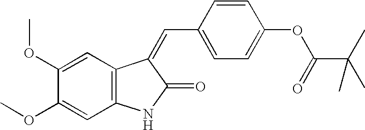 |

G.2 5-tert-Butoxycarbonylamino-pentanoic Acid, (Z)-4-(5,6-dimethoxy-2-oxo-1,2-dihydro-indol-3-ylidenemethyl)phenyl Ester.

The product was recovered in yield 17% as a brown-orange solid from the column chromatography of Example F.2.

M.p.: 115-117° C. ¹H NMR (DMSO-d₆): δ 1.38 (9H, s), δ 1.50 (2H, quint.), δ 1.64 (2H, quint.), δ 2.61 (2H, t), δ 2.96 (2H, q), δ 3.78 (6H, s), δ 6.46 (1H, s), δ 6.84 (1H, t), δ 7.19 (2H, d, J=8.5 Hz), δ 7.40 (1H, s), δ 7.65 (1H, s), δ 8.38 (2H, d, J=8.5 Hz), δ 10.36 (1H, s). HPLC purity: 98.36%. Elemental analysis. (C₂₇H₃₂N₂O₇): found C (65.4%), H, (6.5%), N, (5.62%)—calculated C (65.31%), H, (6.5%), N, (5.64%).

G.3 1-Methyl-1H-pyrrole-2-carboxylic Acid, (Z)-4-(5,6-dimethoxy-2-oxo-1,2-dihydro-indol-3-ylidenemethyl)phenyl Ester.

The product was recovered in 4% yield as a red-orange solid from the column chromatography of Example F.4.

¹H NMR (DMSO-d₆): δ 3.78(6H, s), δ 3.91 (3H, s), δ 6.21 (1H, dd, J=4.02 Hz, J=2.5), δ 6.46 (1H, s), δ 7.13 (1H, d, J=2.5), δ 7.26 (1H, s), δ 7.29 (1H, d, J=4.02), δ 7.30 (2H, d, J=8.5 Hz), δ 7.67 (1H, s), δ 8.42 (2H, d, J=8.5 Hz), δ 10.37 (1H, s). HPLC purity: 98.5%. Elemental analysis. (C₂₃H₂₀N₂O₅):found C (67.23%), H, (5.02%), N, (6.69%)—calculated C (68.31%), H, (4.98%), N, (6.93%).

G.4 1-Adamantyl Carboxylic Acid, (Z)-4-(5,6-dimethoxy-2-oxo-1,2-dihydro-indol-3-ylidenemethyl)phenyl Ester.

¹H NMR (DMSO-d₆): δ 1.74 (6H, m), δ 2.09 (6H, m), δ 2.05 (3H, m), δ 3.78 (6H, s), δ 6.51 (1H, s), δ 7.15 (2H, d J=8.5 Hz), δ 7.40 (1H, s), δ 7.65 (1H, s), δ 8.38 (2H, d, J=8.5), δ 10.36 (1H, s). HPLC purity 99.1%.

G.5 2-(S)-tert-Butoxycarbonylamino Propionic Acid, (Z)-4-(5,6-dimethoxy-2-oxo-1,2-dihydro-indol-3-ylidenemethyl)phenyl Ester.

¹H NMR (DMSO-d₆): δ 3.78 (6H, s), δ 4.25 (1H, q, J=7 Hz), δ 6.46 (1H, s), δ 7.16 (2H, d, J=8.5 Hz), δ 7.4 (1H, s), δ 7.55 (1H, d J=7 Hz), δ 7.65 (1H, s), δ 8.39 (2H, d, J=8.5), δ 10.36 (1H, s). HPLC purity: 96.6%. Elemental analysis. (C₂₅H₂₈N₂O₇): found C (63.92%), H, (5.88%), N, (5.96%)—calculated C (64.09%), H, (6.02%), N, (5.98%).

G.6 Pivalic Acid, (Z)-4-(5,6-dimethoxy-2-oxo-1,2-dihydro-indol-3-ylidenemethyl)phenyl Ester.

¹H NMR (DMSO-d₆): δ 1.3 (9H, s), δ 3.8(6H, s), δ 6.45(1H, s), δ 7.2 (2H, d, J=8.5 Hz), δ 7.4 (1H, s), δ 7.65 (1H, s), δ 7.65 (1H, s), δ 8.4 (2H, d, J=8.5), δ 10.35 (1H, s).

### Example H.1

### (E)-4-(5,6-dimethoxy-2-oxo-1,2-dihydro-indol-3-ylidenemethyl)phenyl, 4-methoxyphenyl Carbonate

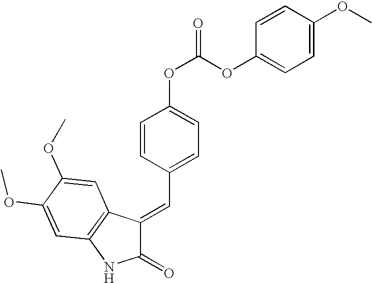

A solution of (E)-3-(4-hydroxybenzylidene)-5,6-dimethoxy-1,3-dihydro-indol-2-one (200 mg, 0.0686 mmoles) in 10 ml of THF, kept under stirring at room temperature, was dropwise added with solution of 4-methoxyphenyl chloroformate (0.1 ml, 0.686 mmoles) in 10 ml of THF. After 4h, the TEA*HCl precipitate was filtered off and mother liquors were concentrated. The resulting crude was purified by silica gel column chromatography (eluent AcOE/n-hexane 8/2) to afford 100 mg (0.223 mmoles, yield 32%) of an orange solid.

M.p.: 165-167° C. ¹H NMR (DMSO-d₆+AcOH-d₄): δ 3.58 (3H, s), δ 3.78 (3H, s), δ 6.51 (1H, s), δ 7.01 (2H, d, J=9 Hz), δ 7.08 (1H, s), δ 7.31 (2H, d, J=9 Hz), δ 7.48 (1H, s), δ 7.55 (2H, d, J=9 Hz), δ 7.85 (2H, d, J=9 Hz), δ 10.33 (1H, s). HPLC purity: 93.8%. Elemental analysis (C₂₅H₂₁NO₇): found C (66.73%), H, (4.77%), N, (3.15%)—calculated C (67.11%), H, (4.73%), N, (3.13%).

### Example H.2

### (Z)-4-(5,6-dimethoxy-2-oxo-1,2-dihydro-indol-3-ylidenemethyl)phenyl, 4-methoxyphenyl Carbonate

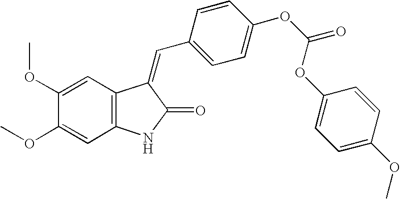

The product was isolated from the column chromatography of Example H.1. 0.04 g (yield 13%) of a red solid were obtained.

M.p.: 176-178° C. ¹H NMR (DMSO-d₆+AcOH-d₄): δ 3.78 (9H, s), δ 6.48 (1H, s), δ 7.01 (2H, d, J=9 Hz), δ 7.32 (2H, d, J=9 Hz), δ 7.41 (1H, s), δ 7.49 (2H, d, J=8.5 Hz), δ 7.69 (1H, s), δ 8.41 (2H, d, J=8.5 Hz), δ 10.33 (1H, s). HPLC purity: 91.6%. Elemental analysis (C₂₄H₂₁NO₇): found C (65.48%), H, (4.90%), N, (3.36%)—calculated C (67.11%), H, (4.73%), N, (3.13%).

### Example H.3

### (E)-4-(5,6-dimethoxy-2-oxo-1,2-dihydro-indol-3-ylidenemethyl)phenyl, n-hexyl Carbonate

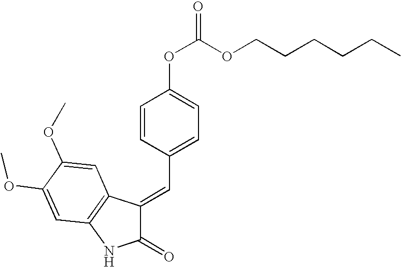

A solution of (E)-3-(4-hydroxybenzylidene)-5,6-dimethoxy-1,3-dihydro-indol-2-one (0.2 g, 0.686 mmole) in THF (10 ml) was dropwise added with a solution of n-hexyl chloroformate (0.112 ml, 0.686 mmole) in THF (10 ml). The mixture was stirred for 4 h at room temperature. The precipitated TEA*HCl was filtered off and filtration waters were concentrated. The resulting residue was purified by silica gel column chromatography, eluting with a AcOEt/n-hexane (9/1) mixture, to afford the product as an orange solid (0.110 g, yield 75%).

M.p.: 139-141° C. ¹H NMR (DMSO-d₆+AcOH-d₄): ¹H NMR (DMSO-d₆+AcOH-d₄): δ 0.88 (3H, t), δ 1.28-1.38 (6H, m), δ 1.68 (2H, quint.), δ 3.58 (3H, s), δ 3.78 (3H, s), δ 4.22 (2H, t), δ 6.51 (1H, s), δ 7.08 (1H, s), δ 7.39 (2H, d, J=9 Hz), δ 7.43 (1H, s), δ 7.76 (2H, d, J=9 Hz), δ 10.35 (1H, s). HPLC purity: 98.34%. Elemental analysis: found C (66.57%), H, (6.4%), N, (3.24%)—calculated C (67.78%), H, (6.40%), N, (3.29%).(C₂₄H₂₇NO₆).

### Example H.4

### (E)-4-(5,6-dimethoxy-2-oxo-1,2-dihydro-indol-3-ylidenemethyl)phenyl, 4-nitrobenzyl carbonate

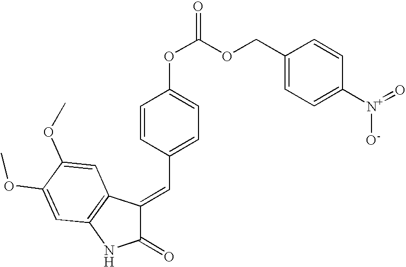

A suspension of (E)-3-(4-hydroxybenzylidene)-5,6-dimethoxy-1,3-dihydro-indol-2-one (1 g, 3.36 mmole), kept under N₂ flow at 0° C., and TEA (0.7 ml, 5.04 mmole) in DCM (50 ml), was dropwise added with a solution of 4-nitrobenzyl-chloroformate (1.08, 5.04 mmole) in DCM (300 ml). The mixture was stirred for 22 h at room temperature. The solid precipitate was recovered by filtration and resuspended in Et₂O (4 ml), then filtered again to afford the desired product as a red solid (1.06 g, yield 69%).

M.p.: 225-227° C. ¹H NMR (DMSO-d₆+AcOH-d₄): δ 3.55 (3H, s), δ 3.78 (3H, s), δ 5.45 (2H, s), δ 6.51 (1H, s), δ 7.06 (1H, s), δ 7.43 (2H, d, J=9.5 Hz), δ 7.44 (1H, s), δ 7.73-7.78 (4H, m), δ 8.28 (2H, d, J=9.5 Hz), 10.31 (1H, s). HPLC purity: 94.6%. Elemental analysis found C (62.43%), H, (4.25%), N, (5.81%)—calculated C (63.028%), H, (4.23%), N, (5.88%).(C₂₄H₂₇NO₆).

### Example H.5

### (E)-4-(5,6-dimethoxy-2-oxo-1,2-dihydro-indol-3-ylidenemethyl)phenyl, 4-nitrophenyl Carbonate

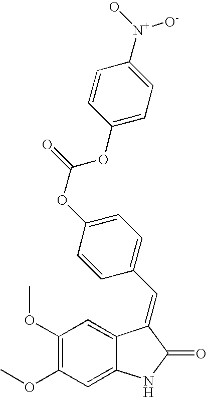

A solution of (E)-3-(4-hydroxybenzylidene)-5,6-dimethoxy-1,3-dihydro-indol-2-one (3 g, 0.0103 moles) and TEA (1.6 ml, 0.0115 moles) in 150 ml of dry dichloromethane, kept under nitrogen stream and at 0° C., was dropwise added with a solution of 4-nitrophenyl chloroformate (2.31 g, 0.0115 moles) in 150 ml of anhydrous dichloromethane. After completion of the addition, stirring was maintained for 12 h. The formed precipitate was filtered and treated with 20 ml of Et₂O. After filtration and drying, 3.4 g (0.0073 moles, yield 71.4%) of a red solid were obtained.

M.p.: 178-180° C. ¹H NMR (DMSO-d₆): δ 3.55 (3H, s), δ 3.7 (3H, s), δ 6.51 (1H, s), δ 7.09 (1H, s), δ 7.43 (1H, s), δ 7.59 (2H, d, J=9 Hz), δ 7.75 (2H, d, J=9 Hz), δ 7.84 (2H, d, J=9 Hz), δ 8.39 (2H, d, J=9 Hz), δ 10.34 (1H, s).

### Example 1.1

### Phenylmethanesulfonic Acid, (E)-4-(5,6-dimethoxy-2-oxo-1,2-dihydro-indol-3-ylidenemethyl)phenyl Ester

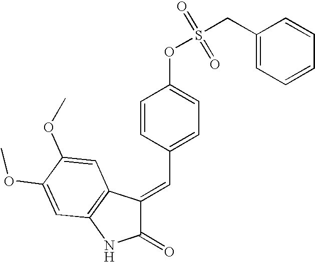

Step 1: Synthesis of Phenylmethanesulfonic Acid, 4-formylphenyl Ester.

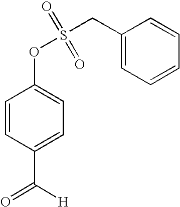

A suspension of 4-hydroxybenzaldehyde (500 mg, 4.012 mmol), in dichloromethane (5 mL), cooled to 0°-5° C., was added with triethylamine (0.62 mL, 1.1 eq.), then phenyl methanesulfonyl chloride (commercially available; CAS No. [1939-99-7]), (841 mg, 1.1 eq.) in dichloromethane was dropwise added. After completion of the reaction (TLC, silica gel, eluent petroleum ether /ethyl acetate 6/4, Rf, 0.6) the solvent was evaporated off under reduced pressure and the residue was extracted with ethyl acetate (20 mL) and water (40 mL). The organic phase was dried over dry Na₂SO₄, and the resulting residue was recrystallized from ethyl acetate (3 mL). The solid was filtered, to obtain 765 mg of product, yield 69%. ¹H NMR (DMSO-d₆): δ 4.58 (2H, s), δ 7.26 (2H, d), δ 7.27 (1H, s), δ 7.44-7.47 (5H, m), δ 7.89 (2H, d), δ 9.99 (1H, s).

Step 2: Phenylmethanesulfonic Acid, (E)-4-(5,6-dimethoxy-2-oxo-1,2-dihydro-indol-3-ylidenemethyl)phenyl Ester.

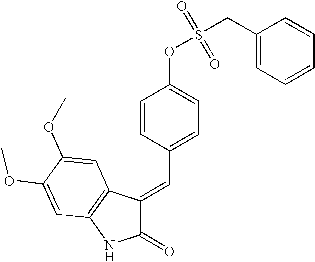

A mixture of phenylmethanesulfonic acid 4-formylphenyl ester from step 1 (343 mg, 1.2 eq.), 5,6-dimethoxy-1,3-dihydro-indol-2-one (200 mg, 1.035 mmol, 1 eq.) and piperidine (10 μl, 0.1 eq.) in ethanol (4 mL) was refluxed for 3 hours, then cooled to crystallize an orange-red solid, which was filtered and dried under vacuum, to obtain 310 mg of product. yield 66%. ¹H NMR (DMSO-d6): δ 3.56 (3H, s), δ 3.79 (3H, s), δ 5.03 (2H, s), δ 6.51 (1H, s), δ 7.01 (1H, s), δ 7.36 (2H, d), δ 7.37-7.45 (5H, m), δ 7.51 (1H, s), δ 7.77 (2H, d), δ 10.36 (1H, s).

Evaluation of c-kit Inhibiting Activity.

The compounds of the invention were tested with a dedicated cell-free kinase assay for the evaluation of the inhibiting activity on tyrosine kinases.

In more detail, the assay was a DELFIA™ (Dissociation Enhanced Time-Resolved Fluorometric Assay) Tyrosine Kinase Assay™ (PerkinElmer), a highly sensitive, reproducible ELISA assay that allows to evaluate the activity of a tyrosine kinase.

Human cKit Tyrosine Kinase Receptor (human cKit RTK-catalytic domain), commercially available from Panvera-Invitrogen (Cat. N^{o}. P3080), was used as the tyrosine kinase.

Screening of potential inhibitors of cKit TKR kinase activity was carried out in polypropylene 96-well plates (U-bottom polypropylene 96-well plates, Greiner). For each experimental point, 25 ng of cKit TKR, 1× cKit Kinase Assay Buffer (20 mM Hepes pH 7.5, 10 mM MnCl₂, 1 mM DTT; Lennartsson J. et al., Oncogene 1999, 18:5546-5553), 150 μM (˜3×Kₘ) of ATP (Promega) as γ-phosphate donor, 200 nM (˜1.5×Kₘ) of biotinylated polyGAT (biotinylated [poly(Glu-Ala-Tyr 6:3:1), PerkinElmer] as γ-phosphate acceptor, 5% (v/v) DMSO (as the solvent for the tested compounds) and H₂O “Molecular Biology Grade” (Sigma) to a final volume of 100 μl have been mixed in each well. After 20 minutes incubation at 30° C., the kinase reactions were quenched by addition of EDTA (final concentration 50 mM), diluted 1:8 in DELFIA Assay Buffer (PerkinElmer) and transferred in 96-well plates evenly coated with streptavidine (PerkinElmer) to immobilize the biotinylated polyGAT (now phosphorylated on tyrosine residues in amount directly proportional to c-Kit TKR activity). After 1 hour incubation at room temperature and three washings with 1×DELFIA Wash Buffer (PerkinElmer), the phosphotyrosine residues of the acceptor were detected with an europium-labelled anti-phosphotyrosine monoclonal antibody (DELFIA Eu-N1 labelled anti-phosphotyrosine antibody PT66, PerkinElmer) and, after a further hour incubation at room temperature and three washings with 1×DELFIA Wash Buffer, overall phosphorylation of biotinylated polyGAT was determined by addition of Enhancement Solution (PerkinElmer) and fluorimetric analysis of the plates (α-Fusion FP HT, PerkinElmer) in the “Time-Resolved Fluorescence” mode.

Results are reported in Table 7

TABLE 7

| Ex. | Name | IC₅₀ (μM) |
|---|---|---|
| | (E)-3-(4-hydroxybenzylidene)-5,6-dimethoxy-1,3- | 2.37 ± 0.68 |
| | diidroindol-2-one | |
| A.1 | Phenylcarbamic acid, (E)-4-(5,6-dimethoxy-2-oxo-1,2- | 1.66 |
| | dihydro-indol-3-ylidenemethyl)phenyl ester | |
| A.3 | Pentyl carbamic acid, (E)-4-(5,6-dimethoxy-2-oxo-1,2- | 0.38 ± 0.16 |
| | dihydro-indol-3-ylidenemethyl)phenyl ester | |
| A.4 | n-Hexyl carbamic acid, (E)-4-(5,6-dimethoxy-2-oxo-1,2- | 0.135 |
| | dihydro-indol-3-ylidenemethyl)phenyl ester | |
| B.1 | (E)-4-Methyl-piperazine-1-carboxylic acid, 4-(5,6- | 4.46 |
| | dimethoxy-2-oxo-1,2-dihydro-indol-3-ylidenemethyl)- | |
| | phenyl ester | |
| B.3 | 4-(2-Hydroxyethyl)piperazine-1-carboxylic acid, (E)-4-(5,6- | 6.74 |
| | dimethoxy-2-oxo-1,2-dihydro-indol-3-ylidenemethyl)phenyl | |
| | ester | |
| B.5 | 4-(1′-Piperidinyl)-1-piperidinecarboxylic acid, (E)-4-(5,6- | 4.68 |
| | dimethoxy-2-oxo-1,2-dihydro-indol-3-ylidenemethyl)phenyl | |
| | ester | |
| B.6 | 4-[2-(2-Hydroxyethoxy)ethyl]piperazine-1-carboxylic acid, | 7.47 |
| | (E)-4-(5,6-dimethoxy-2-oxo-1,2-dihydro-indol-3- | |
| | ylidenemethyl)phenyl ester. | |
| B.7 | 4-Phenylpiperazine-1-carboxylic acid, (E)-4-(5,6- | 2.78 |
| | dimethoxy-2-oxo-1,2-dihydro-indol-3-ylidenemethyl)- | |
| | phenyl ester | |
| C.1 | (E)-4-(5,6-Dimethoxy-2-oxo-1,2-dihydro-indol-3- | 51.80 |
| | ylidenemethyl)phenyl phosphoric acid. | |
| C.2 | (E)-4-(5,6-dimethoxy-2-oxo-1,2-dihydro-indol-3- | 5.09 |
| | ylidenemethyl)phenyl diethyl phosphate | |
| D.1 | (E)3-[4-(2-hydroxyetoxy)benzylidene]-5,6-dimethoxy-1,3- | 3.63 |
| | dihydro-indol-2-one | |
| F.1 | n-Decanoic acid, (E)-4-(5,6-dimethoxy-2-oxo-1,2-dihydro- | 10.16 |
| | indol-3-ylidenemethyl)phenyl ester | |
| F.2 | 5-tert-Butoxycarbonylamino-pentanoic acid, (E)-4-(5,6- | 1.2 |
| | dimethoxy-2-oxo-1,2-dihydro-indol-3-ylidenemethyl)phenyl | |
| | ester | |
| F.3 | [2-(2-Methoxy-ethoxy)-ethoxy]acetic acid, (E)-4-(5,6- | 5.08 |
| | dimethoxy-2-oxo-1,2-dihydro-indol-3-ylidenemethyl)- | |
| | phenyl ester | |
| F.5 | Pyrazine-2-carboxylic acid, (E)-4-(5,6-dimethoxy-2-oxo- | 8.8 |
| | 1,2-dihydro-indol-3-ylidenemethyl)phenyl ester | |
| G.1 | n-Decanoic acid, (Z)-4-(5,6-dimethoxy-2-oxo-1,2-dihydro- | 0.26 |
| | indol-3-ylidenemethyl)phenyl ester | |
| G.2 | 5-tert-Butoxycarbonylamino-pentanoic acid, (Z)-4-(5,6- | 0.77 |
| | dimethoxy-2-oxo-1,2-dihydro-indol-3-ylidenemethyl)- | |
| | phenyl ester | |
| G.3 | 1-Methyl-1H-pyrrole-2-carboxylic acid, (Z)-4-(5,6- | 2.65 |
| | dimethoxy-2-oxo-1,2-dihydro-indol-3-ylidenemethyl)phenyl | |
| | ester | |
| H.1 | (E)-4-(5,6-Dimethoxy-2-oxo-1,2-dihydro-indol-3- | 1.19 |
| | ylidenemethyl)phenyl, 4-methoxyphenyl carbonate | |
| H.2 | (Z)-4-(5,6-Dimethoxy-2-oxo-1,2-dihydro-indol-3- | 0.28 |
| | ylidenemethyl)phenyl, 4-methoxyphenyl carbonate | |

Evaluation of Ret Inhibitory Activity

In order to evidence and quantify inhibitory activity on Ret tyrosine kinase, the most representative compounds of the invention were tested with a dedicated DELFIA kinase assay, using a human tyrosine-kinase receptor (Ret TKR) recombinant form (human Ret TKR-catalytic domain) available from ProQinase as the tyrosine kinase.

The DELFIA Ret Kinase Assay was optimised and carried out according to a procedure similar to the DELFIA cKit Kinase Assay using, for each experimental point, 50 ng of Ret TKR, 1× Ret Kinase Assay Buffer (60 mM Hepes pH 7.5, 3 mM MnCl₂, 3 mM MgCl₂,1.2 mM DTT, 3 μM Na₃VO₄, 2.5 μM PEG₃₃₅₀; data sheet Ret TKR ProQinase), 3 μM (˜3×Kₘ) of ATP (Promega) as γ-phosphate donor, 100 nM (˜3×Kₘ) of biotinylated polyGAT (biotinylated[poly(Glu-Ala-Tyr 6:3:1)], PerkinElmer) as γ-phosphate acceptor, 5% (v/v) DMSO as the solvent for the tested compounds and “Molecular Biology Grade” H₂O (Sigma) to a final volume of 100 μl.

After 10 min. incubation at 30° C., the kinase reactions were worked up as in the DELFIA cKit Kinase Assay.

Results are reported in Table 8.

TABLE 8

| Ex. | Name | IC₅₀ (μM) |
|---|---|---|
| | (E)-3-(4-hydroxybenzylidene)-5,6-dimethoxy-1,3-dihydroindol- | 3.67 |
| | 2-one | |
| A.1 | Phenylcarbamic acid, (E)-4-(5,6-dimethoxy-2-oxo-1,2-dihydro- | 8.39 |
| | indol-3-ylidenemethyl)phenyl ester | |
| A.3 | Pentyl carbamic acid, (E)-4-(5,6-dimethoxy-2-oxo-1,2-dihydro- | 1.78 |
| | indol-3-ylidenemethyl)phenyl ester | |
| A.4 | n-Hexyl carbamic, (E)-4-(5,6-dimethoxy-2-oxo-1,2-dihydro- | 5.73 |
| | indol-3-ylidenemethyl) phenyl ester | |
| B.1 | (E)-4-Methyl-piperazine-1-carboxylic acid, 4-(5,6-dimethoxy- | 13.32 |
| | 2-oxo-1,2-dihydro-indol-3-ylidenemethyl) phenyl ester. | |
| B.3 | 4-(2-Hydroxyethyl)piperazine-1-carboxylic acid, (E)-4-(5,6- | 1.77 |
| | dimethoxy-2-oxo-1,2-dihydro-indol-3-ylidenemethyl)phenyl | |
| | ester. | |
| B.5 | 4-(1′-Piperidinyl)-1-piperidinecarboxylic, (E)-4-(5,6- | 2.17 |
| | dimethoxy-2-oxo-1,2-dihydro-indol-3-ylidenemethyl) phenyl | |
| | ester | |
| B.6 | 4-[2-(2-Hydroxyethoxy)ethyl]piperazine-1-carboxylic acid, (E)- | 6.65 |
| | 4-(5,6-dimethoxy-2-oxo-1,2-dihydro-indol-3- | |
| | ylidenemethyl)phenyl ester. | |
| B.7 | 4-Phenylpiperazine-1-carboxylic acid, (E)-4-(5,6-dimethoxy-2- | 3.23 |
| | oxo-1,2-dihydro-indol-3-ylidenemethyl) phenyl ester | |
| C.1 | (E)-4-(5,6-Dimethoxy-2-oxo-1,2-dihydro-indol-3- | 2.61 |
| | ylidenemethyl)phenylphosphoric acid. | |
| C.2 | (E)-4-(5,6-dimethoxy-2-oxo-1,2-dihydro-indol-3- | 63.54 |
| | ylidenemethyl)phenyldiethyl phosphate | |
| D.1 | (E)-3-[4-(2-hydroxyethoxy)benzylidene]-5,6-dimethoxy-1,3- | 1.12 |
| | dihydro-indol-2-one | |
| F.1 | n-Decanoic acid, (E)-4-(5,6-dimethoxy-2-oxo-1,2-dihydro- | 4.49 |
| | indol-3-ylidenemethyl)phenyl ester | |
| F.2 | 5-tert-Butoxycarbonylamino-pentanoic acid, (E)-4-(5,6- | 1.37 |
| | dimethoxy-2-oxo-1,2-dihydro-indol-3-ylidenemethyl) phenyl | |
| | ester | |
| F.3 | [2-(2-Methoxy-ethoxy)-ethoxy]acetic acid, (E)-4-(5,6- | 3.79 |
| | dimethoxy-2-oxo-1,2-dihydro-indol-3-ylidenemethyl) phenyl | |
| | ester | |
| F.5 | Pirazine-2-carboxylic acid, (E)-4-(5,6-dimethoxy-2-oxo-1,2- | 1.81 |
| | dihydro-indol-3-ylidenemethyl) phenyl ester. | |
| G.1 | n-Decanoic acid, (Z)-4-(5,6-dimethoxy-2-oxo-1,2-dihydro- | 37.70 |
| | indol-3-ylidenemethyl) phenyl ester. | |
| G.2 | 5-tert-Butoxycarbonylamino-pentanoic acid, (Z)-4-(5,6- | 3.45 |
| | dimethoxy-2-oxo-1,2-dihydro-indol-3-ylidenemethyl) phenyl | |
| | ester | |
| G.3 | 1-Methyl-1H-pirrole-2-carboxylic acid, (Z)-4-(5,6-dimethoxy- | 6.38 |
| | 2-oxo-1,2-dihydro-indol-3-ylidenemethyl)phenyl ester. | |
| H.1 | (E)-4-(5,6-dimethoxy-2-oxo-1,2-dihydro-indol-3- | 4.43 |
| | ylidenemethyl) phenyl, 4-methoxyphenylcarbonate | |
| H.2 | (Z)-4-(5,6-dimethoxy-2-oxo-1,2-dihydro-indol-3- | 4.84 |
| | ylidenemethyl)phenyl, 4-methoxyphenylcarbonate | |

Activity on NIH3T3^{RETC634R} Cells

NIH3T3 Murine fibroblasts and the transfected line NIH₃T₃^{MEN2A(C634R)} were treated with increasing concentrations of the compounds of the invention for 72 h. Survival curves were obtained by Coulter Counter cell count. IC₅₀s calculated from said curves are reported in Table 9: the higher sensitivity of the oncoprotein-expressing cell line is evident. The reversal of the morphologically transformed phenotype of NIH₃T₃^{MEN2A} (C634R) cells in the presence of the compounds of the invention was evaluated by contrast phase microscopy after 24 h treatment and photographically documented. The observed effects are reported in Table 9.

TABLE 9

| | | Cont at 72 h | Morphology |
|---|---|---|---|
| Ex. | Name | IC₅₀ (μM) | a 24 h |
| — | (E)-3-(4-hydroxybenzylidene)-5,6- | 3.6 ± 1.6 | REVERSAL |
| | dimethoxy-1,3-diidroindol-2-one | | (from 6 μM) |
| A.3 | Pentyl carbamic acid, (E)-4-(5,6- | 2.2 ± 0.7 | REVERSAL |
| | dimethoxy-2-oxo-1,2-dihydro-indol-3- | | (from 5 μM) |
| | ylidenemethyl)phenyl ester | | |
| B.1 | (E)-4-Methyl-piperazine-1-carboxylic acid, | 2.8 ± 0.5 | REVERSAL |
| | 4-(5,6-dimethoxy-2-oxo-1,2-dihydro-indol- | | (da 10 μM) |
| | 3-ylidenemethyl)phenyl ester | | |
| B.3 | 4-(2-Hydroxyethyl)piperazine-1-carboxylic | 3.1 ± 1.3 | REVERSAL |
| | acid, (E)-4-(5,6-dimethoxy-2-oxo-1,2- | | |
| | dihydro-indol-3-ylidenemethyl)phenyl ester | | |
| B.5 | 4-(1′-Piperidinyl)-1-piperidine-carboxylic | 2.0 ± 0.7 | REVERSAL |
| | acid, (E)-4-(5,6-dimethoxy-2-oxo-1,2- | | (da 5 μM) |
| | dihydro-indol-3-ylidenemethyl)phenyl ester | | |
| B.6 | 4-[2-(2-Hydroxyethoxy)-ethyl]piperazine-1- | 5.6 ± 1.8 | REVERSAL |
| | carboxylic acid, (E)-4-(5,6-dimethoxy-2- | | (da 10 μM) |
| | oxo-1,2-dihydro-indol-3-ylidenemethyl)phenyl ester. | | |
| C.1 | (E)-4-(5,6-Dimethoxy-2-oxo-1,2-dihydro- | 3.9 ± 0.25 | REVERSAL |
| | indol-3-ylidenemethyl) phenyl phosphoric acid | | (from 10 μM) |
| C.2 | (E)-4-(5,6-dimethoxy-2-oxo-1,2-dihydro- | 1.7 ± 0.1 | REVERSAL |
| | indol-3-ylidenemethyl) phenyl diethyl phosphate | | (from 5 μM) |
| D.1 | (E)-3-[4-(2-Hydroxyethoxy)-benzylidene]- | 4.3 ± 0.6 | REVERSAL |
| | 5,6-dimethoxy-1,3-dihydro-indol-2-one | | (da 10 μM) |
| E.2 | Acetic acid, (E)-4-(5,6-dimethoxy-2-oxo- | 1.5 ± 0.05 | REVERSAL |
| | 1,2-dihydro-indol-3-ylidenemethyl)phenyl ester | | (from 5 μM) |
| H.1 | (E)-4-(5,6-dimethoxy-2-oxo-1,2-dihydro- | 3.3 ± 0.03 | REVERSAL |
| | indol-3-ylidenemethyl) phenyl, 4-methoxyphenyl carbonate | | (from 10 μM) |

1. Compounds of general formula (I): 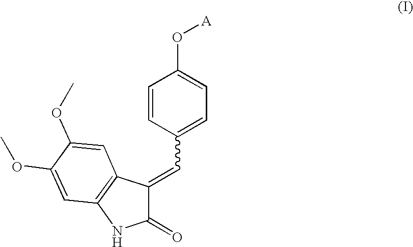
or the pharmaceutically acceptable salts thereof, stereomeric or tautomeric forms, in which:
A is selected from the group consisting of: —R, —C(═O)—R1, —C(═O)—OR2, —C(═X)—NR3R4, —, —P(═O)(OR5)(OR6), —(O═S═O)R7; or A is an optionally protected aminoacyl residue;
R is optionally substituted alkyl, optionally substituted alkenyl, optionally substituted alkynyl;
R1 is H, optionally substituted alkyl, optionally substituted alkenyl, optionally substituted alkynyl, optionally substituted carbocyclyl, optionally substituted heterocarbocyclyl;
R2 is optionally substituted alkyl, optionally substituted alkenyl, optionally substituted alkynyl, optionally substituted carbocyclyl, optionally substituted heterocarbocyclyl;
R3 and R4 are independently H, optionally substituted alkyl, optionally substituted alkenyl, optionally substituted alkynyl, optionally substituted carbocyclyl, optionally substituted heterocarbocyclyl, or R3 and R4, together with the nitrogen atom they are linked to, form a heterocyclic ring which can be optionally substituted;
R5 and R6 are independently H, C1-C6-alkyl, C7-C10-aralkyl;
R7 is C1-C6-alkyl, C7-C10-aralkyl;
X is O, S.

2. Compounds as claimed in claim 1, wherein A is a group —R as defined above.

3. Compounds as claimed in claim 1, wherein A is a group —C(═O)—R1 wherein R1 is as defined above.

4. Compounds as claimed in claim 1, wherein A is a group —C(═O)—OR2 wherein R2 is as defined above.

5. Compounds as claimed in claim 1, wherein A is a group —C(═X)—NR3R4 wherein R3 and R4 are as defined above.

6. Compounds as claimed in claim 1, wherein A is a group —P(═O)(OR5)(OR6) wherein R5 and R6 are as defined above.

7. Compounds as claimed in claim 1, wherein R is 2-hydroxyethyl, 2-aminoethyl, tert-butoxycarbonylmethyl or carboxymethyl.

8. Compounds as claimed in claim 1, wherein the group R1 is methyl, tert-butyl, n-nonyl, adamantyl, 2-(2-methoxy-ethoxy)ethoxymethyl, phenyl, 4-cyano-phenyl, 4-(5-tetrazolyl)-phenyl, 3-pyridyl, 5-oxazolyl, 2-pyrazinyl, 1-methyl-1H-2-pirrolyl, 4-tert-butoxycarbonylaminobutyl, 2(S)-tert-butoxycarbonylaminoethyl.

9. Compounds as claimed in claim 1, wherein R2 is 4-methoxyphenyl, n-pentyl, 4-nitrophenyl.

10. Compounds as claimed in claim 1, wherein one of R3 and R4 is hydrogen and the other is tert-butyl, n-pentyl, n-hexyl, phenyl, 4-methoxy-phenyl.

11. Compounds as claimed in claim 1, wherein R3 and R4 are both 2-hydroxyethyl.

12. Compounds as claimed in claim 1, wherein R3 and R4 taken together form a 4-methyl-piperazinyl, morpholyl, 4-(2-hydroxyethyl)-piperazinyl, 4-(2-(hydroxyethoxyethyl)piperazinyl or 4-(1′-piperidinyl)-piperidinyl ring.

13. Compounds as claimed in claim 1, wherein R5 and R6 are both hydrogen, benzyl or ethyl.

14. Compounds as claimed in claim 1, wherein R7 is benzyl.

15. Pharmaceutical compositions containing a compound of claims 1-14 in admixture with a suitable carrier.

16. A method a) of treatment of subjects suffering from tumors in which proteins with tyrosynokinase activity Met, PDGF-R, FGF-R1, FGF-R3, Kit and oncoproteins of the Ret family are involved, which method comprises administering said subjects with an effective amount of a compound of claims 1-14 and b) of control of tumor invasive process.

17. A method as claimed in claim 16, in which tumors are medullary and papillary carcinoma of the thyroid, pheochromocytoma, parathyroids hyperplasia, multiple myeloma, bladder and cervix carcinomas, glomes, dermatofibrosarcoma protuberans, kidney tumors, stromal tumors of the gastroenteral tract (GIST), lung small cells tumors, seminomas, mastocytosis and acute myeloid leukemia.
